(19) United States
(12) Patent Application Publication
(10) Pub. No.: US 2021/0094994 A1
(43) Pub. Date: Apr. 1, 2021

(54) CAR T CELLS WITH ONE OR MORE INTERLEUKINS

(71) Applicant: Baylor College of Medicine, Houston, TX (US)

(72) Inventors: Andras Heczey, Houston, TX (US); Sai Batra, Houston, TX (US); Purva Rathi, Houston, TX (US)

(21) Appl. No.: 17/050,341

(22) PCT Filed: Apr. 29, 2019
(86) PCT No.: PCT/US2019/029596

§ 371 Date: Oct. 23, 2020

Related U.S. Application Data
(60) Provisional application No. 62/663,410, filed on Apr. 27, 2018.

(51) Int. Cl. C07K 14/54; C12N 5/0783; C07K 14/725; A61K 35/17; C07K 16/30; A61P 35/00

(52) U.S. Cl. CPC C07K 14/54; C12N 5/0636; C07K 14/5443; C07K 2319/00; A61K 35/17; C07K 16/303; A61P 35/00; C07K 14/7051

(57) ABSTRACT

Embodiments of the disclosure encompass methods and compositions related to targeting of tumor antigen-positive cells with therapy using cells that express a chimeric antigen receptor that targets the tumor antigen-positive cells in the presence of None or more interleukins that enhance efficacy of the tumor antigen-specific chimeric antigen receptors. In specific embodiments, the tumor antigen is glypican-3 and the one or more interleukins are EL-15 and EL-21.

This application claims priority to U.S. Provisional Patent Application Ser. No. 62/663,410, filed Apr. 27, 2018, which is incorporated by reference herein in its entirety.

### TECHNICAL FIELD

Embodiments of the disclosure include at least the fields of immunology, cell biology, molecular biology, and medicine, including at least cancer medicine.

### BACKGROUND

Immunotherapy harnesses the body's ability to fight cancer, and while the treatment of B-cell malignancies using CAR T cells has yielded robust complete remission induction rates, treatment of solid tumors with CAR T cells has yielded only modest antitumor responses so far. Thus additional strategies are necessary to enhance CAR T cells. The present disclosure provides solutions to long-felt needs in the art of adoptive cell therapy.

### BRIEF SUMMARY

The present disclosure is directed to methods of targeting tumor antigen-specific cells with cell therapy and directed to measures to enhance the cell therapy in a particular environment. In specific embodiments, the disclosure concerns methods and compositions for the treatment of cancer, including for enhancing cancer therapy in a microenvironment at a group of cancer cells, such as a tumor microenvironment. In particular embodiments, the cell therapy is enhanced with the use of more than one cytokine with the cell therapy. The cell therapy may comprise cells that have been modified to express one or more engineered molecules (Such as antigen receptors) and/or modified to express one or more exogenous molecules.

In particular cases, the disclosure concerns methods and/or compositions for the treatment of cancers in which the cancer cells express glypican 3 (GPC3), for example as a tumor antigen. Although in certain aspects the cancer may be of any kind, in particular cases the cancer is hepatoblastoma, hepatocellular carcinoma, malignant rhabdoid tumors, yok sac tumors, undifferentiated sarcoma of the liver, liposarcoma, Wilm's tumor, or choriocarcinoma. In specific embodiments, the cancer comprises solid tumors. In at least some cases, the cancer is not hepatocellular carcinoma.

In particular embodiments, the disclosure concerns methods and compositions in which a combination of compositions are utilized for the treatment or prevention of cancer, including at least GPC3-positive cancer, as an example. Compositions include T cells that express a GPC3-targeting chimeric antigen receptor (CAR) and one or more compositions that enhance the efficacy of the GPC3-targeting T cells, such as one, two, or more cytokines, including interleukins. Specific cytokines include IL-15 and IL-21, for example. In certain aspects, T cells redirected against GPC3 control the growth of GPC3-expressing cells, including cancer cells, either in vitro or in vivo, e.g., in an individual having a cancer comprising tumor cells that express GPC3. With the addition of one or more cytokines, the cells are more effective against multiple solid tumors than in the absence of the one or more cytokines.

In certain embodiments, the CAR comprises a single chain variable fragment (scFv) specific for a tumor antigen. In specific embodiments, the tumor antigen is GPC3, and in another specific embodiment, the GPC3-specific CAR comprises an scFv that comprises an amino acid sequence that is at least 90%, 91%, 92%, 93%, 94%, 95%, 96%, 97%, 98%, 99%, or 100% identical to the sequence of SEQ ID NO:1.

The particular GPC3-specific CARs encompassed herein may include one or more costimulatory endodomains, such as CD28, 4-1BB, OX40, DAP10, DAP12, CD27, ICOS, or a combination thereof. The CAR may include one or more transmembrane domains, such as one selected from the group consisting of CD3-zeta, CD28, CD8alpha, CD4, and a combination thereof.

In specific embodiments, the T cells may be CD4+ T cells, CD8+ T cells, Treg cells, Th1 T cells, Th2 T cells, Th17 T cells, γδ T cells, Mucosa associated innate T lymphocytes (MALT cells), unspecific T cells, or a population of T cells that comprises a combination of any of the foregoing. The T cells may harbor a nucleic acid that encodes the CAR, a nucleic acid that encodes one or more interleukins, and a nucleic acid that encodes a suicide gene. In some cases, the CAR, one or more interleukins, and the suicide gene are encoded from the same nucleic acid molecule.

In certain embodiments, GPC3-specific CARs transmit signals to activate immune cells through CD3zeta, CD28, and/or 4-1BB pathways, although the intracellular CAR domain could be readily modified to include other signaling moieties.

In specific methods of the disclosure, an individual who has received GPC3-CAR-expressing T cells is receiving, has received, and/or will receive an additional cancer treatment, such as chemotherapy, immunotherapy, radiation, surgery, hormone therapy, or a combination thereof.

In particular embodiments, cells of the disclosure are not a Natural Killer (NK) cell or an NKT cell.

Embodiments of the disclosure include an isolated T cell, comprising (a) a chimeric antigen receptor that targets a tumor antigen, and (b) one or both of: (i) at least one recombinant interleukin (IL), and (ii) induced expression of at least one endogenous IL, wherein the interleukin is IL-7, IL-2, IL-12, IL-15, IL-21, IL-18 or a combination thereof. The interleukin may be at least IL-15 and/or at least IL-21. The chimeric antigen receptor may be expressed from a recombinant nucleic acid, such as a vector, including a viral vector (adenoviral vector, lentiviral vector, retroviral vector, or adeno-associated viral vector) or non-viral vector (plasmid or nanoparticle, for example). In some cases, IL-15, IL-21, or a combination thereof are expressed from a recombinant nucleic acid and/or from an endogenous gene that is under the transcriptional control of a recombinantly modified promoter region. In specific cases the recombinant IL-15, IL-21, or combination thereof are expressed from a recombinant nucleic acid, such as a vector, including a viral vector or a non-viral vector. In specific embodiments, the tumor antigen-specific CAR is expressed from a recombinant nucleic acid, such as a vector. The recombinant nucleic acid from which the tumor antigen-specific CAR is expressed may or may not be the same molecule as the recombinant nucleic acid from which one or more interleukins are expressed. In some cases, the nucleic acid comprises a cleavable linker between the tumor antigen-specific CAR and the one or more interleukins.

Any tumor antigen-specific CAR may comprise one, two, three, or costimulatory domains, such as a costimulatory domain is selected from the group consisting of CD28, 4-1BB, OX40, DAP10, DAP12, CD27, ICOS, and a combination thereof.

In cases where there is induced expression of at least one endogenous IL in the cell, the induced expression may be from recombinant genome editing of at least one regulatory region of the endogenous IL, and the recombinant genome editing may utilize Zinc finger nucleases (ZFNs), Transcription Activator-Like Effector Nucleases (TALENs), the CRISPR/Cas9 system, and/or engineered meganuclease re-engineered homing endonucleases.

Any T cells of the disclosure may be virus-specific T cells, such as wherein the virus is BK Virus, Human Herpesvirus 6, Cytomegalovirus, Hepatitis B virus, Hepatitis C virus, Epstein-Barr Virus, or Adenovirus.

Embodiments of the disclosure include an isolated population of cells, comprising a plurality of any one of the cells encompassed by the disclosure. In some cases, the majority of cells in the population are the particular T cells encompassed by the disclosure. In certain cases greater than 70, 75, 80, 85, 90, 91, 92, 93, 94, 95, 96, 97, 98, or 99% of cells in the population are the cells encompassed by the disclosure. There are compositions that comprise the population of cells encompassed by the disclosure, and the composition may be in a pharmaceutically acceptable excipient. The population of cells may be in a solution that is sterile, nonpyogenic, and isotonic. The composition may or may not be frozen.

In one embodiment, there is a method of inhibiting proliferation and/or activity of tumor antigen-positive cells in an individual, comprising the step of providing to the individual a therapeutically effective amount of a plurality of the cells encompassed by the disclosure. The tumor antigen may or may not be GPC3. In specific cases, including when the tumor antigen is GPC3, the cancer cells are hepatocellular carcinoma cells, liver cancer cells, embryonal sarcoma cells, rhabdoid tumor cells, Wilms tumor cells, choriocarcinoma cells, or yolk sac tumor cells. The individual may be receiving, has received and/or will receive one or more additional cancer therapies. The individual may have been diagnosed with or suspected of having hepatoblastoma, hepatocellular carcinoma, malignant rhabdoid tumors, yok sac tumors, undifferentiated sarcoma of the liver, liposarcoma, Wilm's tumor, or choriocarcinoma. In any method of the disclosure, the cells may be provided systemically or locally, for example by injection, including at a tumor site(s). The cells may be provided to the individual more than once.

In one embodiment, there is a method of enhancing a T cell therapy of any kind, comprising the step of modifying the T cells to express: (a) recombinant (that includes transgenic) IL-15 and IL-21, (b) induced expression of endogenous IL-15 and IL-21, (c) both (a) and (b), or (d) recombinant IL-15 or IL-21, and induced expression of endogenous IL-21 or IL-15, respectively, wherein after modifying at least some of the T cells are protected from apoptosis following exposure to cancer cells and/or wherein the T cells have enhanced in vivo expansion and persistence compared to T cells lacking the modifying step. In specific embodiments, the T cell therapy comprises T cells modified to express one or more engineered antigen receptors (such as synthetic or produced by the hand of man, such as with recombinant technology), such as a chimeric antigen receptor, a T cell receptor, or both. The T cell therapy may comprise T cells modified to express a chimeric antigen receptor that targets GPC3, as one example. The production of the cells may or may not be automated.

In some embodiments, there is a method of protecting T cells of a T cell therapy from apoptosis upon exposure to cancer cells, comprising the step of modifying the T cells to express: (a) recombinant IL-15 and IL-21, (b) induced expression of endogenous IL-15 and IL-21, (c) both (a) and (b), or (d) recombinant IL-15 or IL-21, and induced expression of endogenous IL-21 or IL-15, respectively, wherein following the modifying step the T cells are protected from apoptosis upon exposure to cancer cells.

In certain embodiments, there is a method of increasing the expansion and persistence of T cell therapy, comprising the step of modifying the T cells to express: (a) recombinant IL-15 and IL-21, (b) induced expression of endogenous IL-15 and IL-21, (c) both (a) and (b), or (d) recombinant IL-15 or IL-21, and induced expression of endogenous IL-21 or IL-15, respectively, wherein following the modifying step the T cells have increased expansion and persistence compared to T cells without the modifying.

In particular embodiments, there is a method of inducing TCF-1 expression in T cells of a T cell therapy, comprising the step of modifying the T cells to express: (a) recombinant IL-15 and IL-21, (b) induced expression of endogenous IL-15 and IL-21, (c) both (a) and (b), or (d) recombinant IL-15 or IL-21, and induced expression of endogenous IL-21 or IL-15, respectively, wherein following the modifying step the T cells have increased expression of TCF-1.

The foregoing has outlined rather broadly the features and technical advantages of the present disclosure in order that the detailed description that follows may be better understood. Additional features and advantages will be described hereinafter which form the subject of the claims herein. It should be appreciated by those skilled in the art that the conception and specific embodiments disclosed may be readily utilized as a basis for modifying or designing other structures for carrying out the same purposes of the present designs. It should also be realized by those skilled in the art that such equivalent constructions do not depart from the spirit and scope as set forth in the appended claims. The novel features which are believed to be characteristic of the designs disclosed herein, both as to the organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present disclosure.

### BRIEF DESCRIPTION OF THE DRAWINGS

For a more complete understanding of the present disclosure, reference is now made to the following descriptions taken in conjunction with the accompanying drawing, in which:

FIGS. 1A-1D. Generation of GPC3-CAR T cells that co-express IL-21 and IL-15. (FIG. 1A) Schematic of retroviral constructs encoding GPC3-CAR (GBBz) with and without IL-15 and/or IL-21. FIG. 1B) CAR expression in T cells transduced (on Day 3 post stimulation with plate bound antibody) using retroviral vectors containing the indicated GPC3-CAR constructs as measured by flow cytometry (on Days 10-14). Data from one representative donor and summary for eight independent donors is shown (mean+SD). IL-15 (FIG. 1C) and IL-21 (FIG. 1D) levels produced by the indicated T cell groups at baseline (left) or after stimulation with GPC3-positive Huh-7 cells (right, E:T=1:1, +72 hrs) as measured by ELISA (mean±SD, n=8). One-way ANOVA. * p<0.05, ** p<0.01, ***p<0.001

FIGS. 2A-2C. Co-expression of IL-15 and/or IL-21 maintains GPC3-specific tumor cell killing but alters effector cytokine release in GPC3-CAR T cells. (FIG. 2A) GPC3-CAR T cells were co-cultured with 51Cr-labeled GPC3-positive (Huh-7, Hep3B, G401, A549-GPC3) and negative (A549) target cells at the indicated effector-to-target (E:T) ratios. 51Cr-release was detected after four hours as a measure of GPC3-CAR T cytotoxicity (mean±SEM, n=4). (FIG. 2B) Indicated T cell groups were cultured with Huh-7 tumor cells at a 1:1 ratio and concentration of indicated effector cytokines released into the supernatant (+24 hrs) was measured by Luminex assay (n=6). Comparison with two-way ANOVA. (FIG. 2C) Surface expression of CD4 (top) and CD8 (bottom) populations within CAR-positive cells at baseline (left) and after two consecutive stimulations with Huh-7 cells (right, E:T=1:1) as measured by FACS (mean±SD, n=6), one-way ANOVA. *p<0.05, ** p<0.01, ***p<0.001.

FIGS. 3A-3C. Co-expressing IL-21 and IL-15 enhances expansion, enriches for less differentiated subsets, and reduces the apoptosis rate of GPC3-CAR T cells. (FIG. 3A) GPC3-CAR T cells were co-cultured with HCC cells at a 1:1 ratio and re-plated every 3-4 days as indicated with fresh HCC cells in the absence of exogenous cytokines (mean+SEM, n=3). One-way ANOVA followed. (FIG. 3B) Phenotype of GPC3-CAR T cells as measured by surface expression of CD45RO and CD62L after manufacture. Shown are representative flow plots and summary data for indicated CAR T cell groups (mean+SEM, n=4, asterisks indicate significant differences from GBBz). Tscm/Tn: CD45RO−/CD62L+, Tcm: CD45RO+/CD62L+, Tem: CD45RO+/CD62L−, Teff: CD45RO−/CD62L−. FIG. 3C) GPC3-CAR T cells were stimulated once (day 2) or three times (day 9) with HCC cells at a 1:1 ratio and rate of apoptosis was evaluated by staining for annexin V. Representative flow plots and summary bargraph for indicated CAR T cell groups (mean±SEM, n=4). Data in FIGS. 3B and 3C were analyzed using two-way ANOVA. *p<0.05, **p<0.01, ***p<0.001.

FIGS. 4A-4D. Co-expression of IL-21 and IL-15 alters global gene expression patterns in GPC3-CAR T cells and TCF-1 is maintained at the highest level in CAR T cells co-expressing both IL-15 and IL-21. (FIG. 4A) Heat maps showing fold expression changes for top 20 genes with most increase or decrease in expression and reaching significance versus GBBz T cells (arranged with respect to 21.15.GBBz vs GBBz), as measured three days after stimulation with HCC cells. (FIG. 4B-FIG. 4D) TCF-1 protein expression within CD4 and CD8 GPC3-CAR T cells as measured by intracellular flow cytometry. A representative histogram (FIG. 4B) and combined results showing percentage (FIG. 4C) and mean fluorescence intensity (MFI, FIG. 4D) of TCF-1+ cells (mean+SD, n=4). Two-way ANOVA, * p<0.05, ** p<0.01, ***p<0.001.

FIGS. 5A-5G. Co-expression of IL-15 and IL-21 enhances in vivo expansion, persistence, and antitumor activity of GPC3-CAR T cells. (FIG. 5A) Schematic of in vivo evaluation scheme for GPC3-CAR T cell persistence. NSG mice (n=5) were injected with 2×106 Huh-7 cells followed by 2×106 Ffluc+ CAR T cells two weeks later. (FIG. 5B) Monitoring of bioluminescent GPC3-CAR T cells at indicated time points post-injection. (FIG. 5C) GPC3-CAR T cell bioluminescence counts (mean±SEM) over experimental time course. (FIG. 5D) Ratio of CD4 and CD8 GPC3-CAR T cells relative to mouse CD45-expressing cells in splenic tissue on day 18 as measured by flow cytometry. (mean+SD, n=4-5/GPC3 CAR T group). (FIG. 5E) Schematic of in vivo evaluation scheme for GPC3-CAR T cell antitumor activity. NSG mice (n=5-8/GPC3 CAR T group) were injected with 2×106 Huh-7/FfLuc cells followed by 0.5×106 CART cells on day 7. (FIG. 5F) Weekly monitoring of bioluminescent Huh-7 tumor cells. FIG. 5G) Kaplan-Meier survival analysis of tumor-bearing mice pictured in (FIG. 5F) Data in FIG. 5C and FIG. 5D were analyzed using one-way ANOVA. Survival was estimated by the Kaplan-Meier method and compared by the Gehan-Breslow-Wilcoxon test. *p<0.05, ** p<0.01, *** p<0.001.

FIGS. 6A-6B. GPC3-CAR T cells co-expressing IL-21 and/or IL-15 do not undergo autonomous growth or increase peripheral blood concentrations of either cytokine in vivo. (FIG. 6A) PBMCs were stimulated with plate bound anti-CD3/anti-CD28 antibodies and transduced. 0.5×10⁶ generated CAR T cells were cultured in the absence of exogenous cytokines and were split every 2-4 days as needed to maintain optimal culture conditions. CAR T cell viability was assessed three times a week and cell survival in number of days post-stimulation is shown (n=3). (FIG. 6B) Peripheral blood levels of IL-15 and IL-21 are shown from mice serum collected 15 days post-CAR T injection for indicated treatment groups (Mean+SD, n=5). One-way ANOVA.

FIGS. 7A-7E. CAR T cells only produce effector cytokines upon stimulation with GPC3-positive HCC cells. Production of effector cytokines (FIG. 7A) GM-CSF, (FIG. 7B) IL-13, (FIG. 7C) IFN-γ, (FIG. 7D) IL-2, and (FIG. 7E) TNF-α as measured by Luminex following stimulation with either GPC3-negative A549 or GPC3-positive Huh-7 cells. Summary data for n=6 (Huh-7) and n=4 (A549) independent donors is shown (mean+SD). Two sample t-test. Asterisks indicate significant differences for each CAR T cell group between A549 and Huh-7 co-culture conditions; **p<0.01, ***p<0.001.

FIGS. 8A-8C. Co-expression of IL-15 alone or with IL-21 increases CD8+ population, but does not influence T cell memory subset composition of GPC3-CAR T cells after tumor cell killing. (FIG. 8A) Representative dot plot showing expression of CD4 and CD8 by GPC3-CAR T cells after two consecutive stimulations with Huh-7 cells. (FIG. 8B-FIG. 8C) Phenotype of GPC3-CAR T cells as measured by surface expression of CD45RO and CD62L in (FIG. 8B) CD4 and (FIG. 8C) CD8 GPC3-CAR T cell subsets following one (day 2) or two (day 5) stimulations with Huh-7 cells (mean+SEM, n=4). Tscm/Tn: CD45RO−/CD62L+, Tcm: CD45RO+/CD62L+, Tem: CD45RO+/CD62L−, Teff: CD45RO−/CD62L−. One-way ANOVA.

FIGS. 9A-9C. Exhaustion marker expression in GPC3-CAR T cells before and after stimulation. Surface expression of (FIG. 9A) LAG-3, (FIG. 9B) TIM-3, and (FIG. 9C) PD-1 were assessed by flow cytometry post-manufacture (baseline) and after two stimulations with tumor cells (MFI+SD, n=3). Only MFI changes >2 fold were considered. Two-way ANOVA. *p<0.05, **p<0.01, ***p<0.001 (asterisks represent comparison to GBBz T cell group).

FIGS. 10A-10F. GPC3-CAR T cell gene expression profiles before and after stimulation with HCC cells. Volcano plots showing log 2 fold changes in expression for genes post-manufacture (baseline, FIG. 10A-FIG. 10C) or after tumor cell killing with Huh-7 cells (post-stimulation, day 3, FIG. 10D-FIG. 10F) using the Nanostring™ immuno-oncology panel. Panels show comparisons of gene expression in 15.GBBz (FIG. 10A, FIG. 10D), 21.GBBz (FIG. 10B, FIG. 10E), and 21.15.GBBz (FIG. 10C, FIG. 10F) compared to GBBz. Adjusted p-values are represented by lines across volcano plots. Green arrow: tcf7; red arrow: bcl2.

FIG. 11. GPC3-CAR T cell expression heat maps for select genes at baseline. Heat map showing fold expression changes for top 20 genes with most increase or decrease in expression and reaching significance versus GBBz T cells at baseline (arranged with respect to 21.15.GBBz vs GBBz). Multiple comparisons were performed after using Benjamini-Hochberg correction.

FIG. 12. GPC3-CAR T cells are detected in the peripheral blood of tumor-bearing mice 15 days after adoptive transfer. Frequencies of CD4 and CD8 GPC3-CAR T cells in the peripheral blood of treated mice were measured by flow cytometry 15 days after injection. Ratio to mouse CD45 expressing cells is shown (mean+SD, five mice per group). One-way ANOVA, ** p<0.01, *** p<0.001.

FIG. 13A-13B. 15.GBBz T cells generate a comparable anti-tumor response to 21.15.GBBz in malignant rhabdoid tumor (MRT, G401) xenograft-bearing mice. NSG mice (n=4-8) were injected with 5×10⁶ Ffluc+G401 cells followed by 5×10⁵ GPC3-CAR T cells on day 14. (FIG. 13A) Weekly monitoring of bioluminescent G401 tumor cells. (FIG. 13B) Tumor bioluminescence counts over time for each group. Dashed lines represent bioluminescence of each animal, solid lines represent mean bioluminescence for the indicated treatment group.

FIGS. 14A-14B. 15.GBBz T cells generate a comparable anti-tumor response to 21.15.GBBz T cells at a 2×10⁶ dose in HCC xenograft-bearing mice. NSG mice (n=5) were injected with 2×106 Ffluc+Huh-7 cells followed by 2×106 GPC3-CAR T cells on day 14. (FIG. 14A) Weekly monitoring of bioluminescent Huh-7 tumor cells. (FIG. 14B) Tumor bioluminescence counts over time for each group. Dashed lines represent bioluminescence of each animal, solid lines represent mean bioluminescence for the indicated treatment group.

### DETAILED DESCRIPTION

As used herein the specification, “a” or “an” may mean one or more. As used herein in the claim(s), when used in conjunction with the word “comprising”, the words “a” or “an” may mean one or more than one. As used herein “another” may mean at least a second or more. In specific embodiments, aspects of the subject matter may “consist essentially of” or “consist of” one or more elements or steps of the subject matter, for example. Some embodiments of the subject matter may consist of or consist essentially of one or more elements, method steps, and/or methods of the subject matter. It is contemplated that any method or composition described herein can be implemented with respect to any other method or composition described herein.

Throughout this specification, unless the context requires otherwise, the words “comprise”, “comprises” and “comprising” will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase and limited to other elements that do not interfere with or contribute to the activity or action specified in the disclosure for the listed elements. Thus, the phrase “consisting essentially of” indicates that the listed elements are required or mandatory, but that no other elements are optional and may or may not be present depending upon whether or not they affect the activity or action of the listed elements.

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present invention. Thus, the appearances of the foregoing phrases in various places throughout this specification are not necessarily all referring to the same embodiment. Furthermore, the particular features, structures, or characteristics may be combined in any suitable manner in one or more embodiments.

The term “subject,” as used herein, generally refers to an individual having a biological sample that is undergoing processing or analysis and, in specific cases, has one or more microbiomes associated therewith. A subject can be an animal or plant. The subject can be any organism or animal subject that is an object of a method or material, including mammals, e.g., humans, laboratory animals (e.g., primates, rats, mice, rabbits), livestock (e.g., cows, sheep, goats, pigs, turkeys, and chickens), household pets (e.g., dogs, cats, and rodents), horses, and transgenic non-human animals. The subject can be a patient, e.g., have or be suspected of having a disease (that may be referred to as a medical condition), such as one or more infectious diseases, one or more genetic disorders, one or more cancers, or any combination thereof. The disease may be pathogenic. The subject may being undergoing or having undergone antibiotic treatment. The subject may be asymptomatic. The subject may be healthy individuals. The term “individual” may be used interchangeably, in at least some cases. The “subject” or “individual”, as used herein, may or may not be housed in a medical facility and may be treated as an outpatient of a medical facility. The individual may be receiving one or more medical compositions via the internet. An individual may comprise any age of a human or non-human animal and therefore includes both adult and juveniles (i.e., children) and infants and includes in utero individuals. It is not intended that the term connote a need for medical treatment, therefore, an individual may voluntarily or involuntarily be part of experimentation whether clinical or in support of basic science studies.

### I. General Embodiments

The disclosure concerns methods and compositions for targeting of tumor antigen-positive cells using cell therapy including in a manner wherein the efficacy of the cell therapy is enhanced. In particular embodiments, cell therapy is enhanced because of the use of one or more compositions that facilitate therapeutic activity at a tumor microenvironment. In specific embodiments, the efficacy of the cell therapy is enhanced by the use of one or more cytokine compositions. In particular embodiments, the efficacy of the cell therapy is enhanced by the use of two or more cytokine compositions, and this enhancement exceeds the efficacy that occurs with the use of just one cytokine.

The present disclosure in some embodiments encompasses a combinatorial approach to treating cancer, including cancer in a solid tumor microenvironment. The combinatorial approach allows for efficacy at solid tumors when compared to other approaches at solid tumors (although the disclosure also encompasses treatment of non-solid cancers). The combinatorial approach provides for greater anti-tumor activity compared to activity with separate use of the components of the combination. In particular cases, the combination of components imparts an additive effect for the combination, whereas in other cases the combination of components provides a synergistic effect. In specific cases, additive effect refers to the sum of the outcomes if each component of the combination is used separately, and the synergistic effect refers to greater than the sum of the outcomes if each component is used separately.

In particular embodiments, the treatment encompassed by the disclosure includes at least cell therapy, particular T cell therapy. As part of the therapy, the T cells are utilized in conjunction with one or more particular cytokines. Although the T cells may comprise the one or more particular cytokines, in some embodiments, the T cells do not comprise the one or more particular cytokines and the T cells and the cytokine(s) may be used as separate compositions or have separate sources. In such cases they may be administered or otherwise provided at substantially the same time.

In certain embodiments, T cells that express a chimeric antigen receptor (CAR) that targets a particular tumor antigen is utilized with one or more recombinant interleukins (IL) and/or where the expression of at least one endogenous IL in the T cells themselves are induced at a level above normal for the T cells. In specific aspects, the particular tumor antigen is glypican-3 (GPC3) and the CAR is a GPC3-specific CAR. Examples of other tumor cell antigens to which the CAR may be directed include at least 5T4, 8H9, αvβδ integrin, BCMA, BTLA, B7-H3, B7-H6, CAIX, CA9, CD19, CD20, CD22, CD30, CD33, CD38, CD44, CD44v6, CD44v7/8, CD70, CD123, CD138, CD171, CEA, CSPG4, EGFR, EGFR family including ErbB2 (HER2), EGFRvIII, EGP2, EGP40, ERBB3, ERBB4, ErbB3/4, EPCAM, EphA2, EpCAM, folate receptor-a, FAP, FBP, fetal AchR, FR□, GD2, G250/CAIX, GD3, Glypican-2, Glypican-3 (GPC3), Her2, HLA-A1+MAGE1, HLA-A1+NY-ESO-1, IL-11Ralpha, IL-13Ralpha2, Lambda, Lewis-Y, Kappa, KDR, Melanoma-associated antigen (MAGE), MCSP, Mesothelin, Muc1, Muc16, NCAM, NKG2D Ligands, NY-ESO-1, Preferentially expressed antigen of melanoma (PRAME), PSC1, PSCA, PSMA, ROR1, SP17, Survivin, TAG72, TEMs, carcinoembryonic antigen, HMW-MAA, AFP, CA-125, ETA, Tyrosinase, MAGE, laminin receptor, HPV E6, E7, BING-4, Calcium-activated chloride channel 2, Cyclin-B1, 9D7, EphA3, Telomerase, SAP-1, BAGE family, CAGE family, GAGE family, MAGE family, SAGE family, XAGE family, NY-ESO-1/LAGE-1, PAME, SSX-2, Melan-A/MART-1, GP100/pmel17, TRP-1/-2, P. polypeptide, MC1R, Prostate-specific antigen, β-catenin, BRCA1/2, CML66, Fibronectin, MART-2, TGF-βRII, or VEGF receptors (e.g., VEGFR2)

In particular embodiments, co-expressing homeostatic cytokines in CAR T cells are utilized as an effective strategy, as the solid tumor microenvironment lacks the cytokine support needed for efficient CAR T cell activation and survival. Interleukin-15 (IL-15) and IL-21 are examples of immunomodulatory cytokines supporting T cell expansion, persistence, survival and function. As demonstrated herein, IL-15 and IL-21 can act synergistically to promote T cell expansion and function. In preclinical models of B-cell malignancies, CD19-specific CAR T cells expressing IL-15 or IL-21 alone have shown superior antitumor activity, but the utility of co-expressing IL-15 and/or IL-21 in CAR T cells is yet to be established, including with respect to solid tumors. The results provided herein are evidence for the first time that the co-expression of more than one cytokine allows for enhanced antitumor properties of CAR T cells with this strategy.

### II. T Cells

The present disclosure includes at least T cells that are administered to an individual in need thereof. The T cells of the disclosure may be modified in one or more than one manner. The T cells may express at least one non-natural molecule that is a receptor for an antigen that is present on the surface of one or more types of cells. The T cells, in particular embodiments, include T cells that are not found in nature because they are engineered to comprise or express at least one synthetic molecule that is not found in nature. In specific embodiments, the non-natural T cells are engineered to express at least one chimeric antigen receptor (CAR), including a CAR that targets a specific tumor antigen, such as glypican-3 (GPC3), for example. Cells of the disclosure include T cells that express a GPC3-specific CAR. In certain embodiments, the cells are not NK cells or NKT cells. In specific embodiments, the T cells may be CD4+ T cells, CD8+ T cells, Treg cells, Th1 T cells, Th2 T cells, Th17 T cells, γδT cells, Mucosa associated Innate T lymphocytes (MAIT cells), unspecific T cells, or a population of T cells that comprises a combination of any of the foregoing. In particular embodiments, the cells are isolated, including isolated away from a natural setting, such as isolated away from a mammalian body. Following the isolation, the cells are engineered by the hand of man to comprise at least one non-natural molecule.

In addition to the T cells expressing a tumor antigen-specific CAR, they may also express or have increased expression of one or more cytokines. The increased expression of the cytokine(s) may refer to an increased level with respect to a cell that has not been modified to have increased expression of one or more cytokines. In specific embodiments, the T cells comprise a tumor antigen-specific CAR and increased expression of one or more cytokines. In specific cases, tumor antigen-specific CAR T cells have one or both of (i) a recombinant interleukin (IL) and (ii) induced endogenous IL expression. The T cells have increased levels of one or more interleukins because of induced expression of endogenous genes in T cells and/or they have increased levels of one or more interleukins because they are transduced with a transgene encoding the interleukin(s). In specific cases the T cells have increased expression of IL-7, IL-2, IL-12, IL-15, IL-21, and/or IL-18, and this increased expression induces enhanced antitumor properties of the modified T cells.

As used herein, the terms “cell,” “cell line,” and “cell culture” may be used interchangeably. All of these terms also include their progeny, which is any and all subsequent generations. It is understood that all progeny may not be identical due to deliberate or inadvertent mutations. In the context of expressing a heterologous nucleic acid sequence, “host cell” refers to a eukaryotic cell that is capable of replicating a vector and/or expressing a heterologous gene encoded by a vector. A host cell can, and has been, used as a recipient for vectors. A host cell may be “transfected” or “transformed,” which refers to a process by which exogenous nucleic acid is transferred or introduced into the host cell. A transformed cell includes the primary subject cell and its progeny. As used herein, the terms “engineered” and “recombinant” cells or host cells are intended to refer to a cell into which an exogenous nucleic acid sequence, such as, for example, a vector, has been introduced. In some cases, the exogenous nucleic acid is introduced to increase expression of an endogenous nucleic acid, such as a heterologous promoter that regulates expression of an endogenous nucleic acid. Therefore, recombinant cells are distinguishable from naturally occurring cells which do not contain a recombinantly introduced nucleic acid. In embodiments of the disclosure, a host cell is a T cell, including a cytotoxic T cell (also known as TC, Cytotoxic T Lymphocyte, CTL, T-Killer cell, cytolytic T cell, CD8+ T-cells or killer T cell.

In some embodiments, it is contemplated that RNAs or proteinaceous sequences may be co expressed with other selected RNAs or proteinaceous sequences in the same cell, such as the same T cell. Co-expression may be achieved by co-transfecting the T cell with two or more distinct recombinant vectors; in such cases, one vector may encode one or more CARs and a second vector may encode one or more cytokines. Different cytokines may be expressed from different vectors, in certain cases. Alternatively, a single recombinant vector may be constructed to include multiple distinct coding regions for RNAs, which could then be expressed in CTLs transfected with the single vector. The single vector may encode the CAR and one or more cytokines.

Some vectors may employ control sequences that allow it to be replicated and/or expressed in both prokaryotic and eukaryotic cells. One of skill in the art would further understand the conditions under which to incubate all of the above described host cells to maintain them and to permit replication of a vector. Also understood and known are techniques and conditions that would allow large-scale production of vectors, as well as production of the nucleic acids encoded by vectors and their cognate polypeptides, proteins, or peptides.

The cells can be autologous cells, syngeneic cells, allogenic cells and even in some cases, xenogeneic cells.

In many situations one may wish to be able to kill the tumor antigen-specific T cells, where one wishes to terminate the treatment, the cells become neoplastic, in research where the absence of the cells after their presence is of interest, or another event, for example. For this purpose, one can provide for the expression of certain gene products in which one can kill the modified cells under controlled conditions, such as inducible suicide genes. In certain embodiments, the suicide gene is caspase-9 or HSV thymidine kinase, for example. An inducible suicide gene may be used to reduce the risk of direct toxicity and/or uncontrolled proliferation, for example. In specific aspects, the suicide gene is not immunogenic to the host harboring the polynucleotide or cell. A certain example of a suicide gene that may be used is caspase-9 or caspase-8 or cytosine deaminase. Caspase-9 can be activated using a specific chemical inducer of dimerization (CID), for example. Thymidine kinase-based suicide systems may be utilized.

### III. Immunomodulatory Cytokines

In particular embodiments of the disclosure, one or more, and preferably two or more, immunomodulatory cytokines that support T cell expansion, persistence, survival and function are utilized in compositions and methods of the disclosure. In specific embodiments, the cytokines are one or more, or two or more, interleukins. In particular embodiments, one or more of the following interleukins are utilized with or in or are expressed from tumor antigen-specific CAR T cells to induce enhanced antitumor properties: IL-7, IL-2, IL-12, IL-15, IL-21, and/or IL-18. One, two, three, four, or all of IL-7, IL-2, IL-12, IL-15, IL-21, and IL-18 may be utilized. In specific embodiments, a combination that comprises, consists of, or consists essentially of IL-15 and IL-21 are utilized in methods and compositions of the disclosure. As examples only, particular combinations of cytokines comprise, consist of, or consist essentially of IL-7 and IL-2; IL-7 and IL-15; IL-7 and IL-21; IL-7 and IL-18; IL-2 and IL-15; IL-2 and IL-21; IL-2 and IL-18; IL-15 and IL-21; IL-15 and IL-18; and IL-21 and IL-18. Specific combinations of cytokines comprise, consist of, or consist essentially of IL-15, IL-21, and IL-7; IL-15, IL-21, and IL2; or IL-15, IL-21, and IL-18.

The manner in which the cytokine is delivered to the microenvironment of cancer cells in an individual in conjunction with the tumor antigen-specific CAR may be in the form of the cell expressing the CAR (or recombination T cell receptor, for example). The cytokine may be expressed from the cell from a recombinant vector as with a transduced cell harboring the vector. In other cases, the cytokine is endogenous to the T cell but the T cell is modified to increase the level of expression of the cytokine above the normal level of expression of the cytokine in the T cell. In such cases, the genome of the T cell may be modified to incorporate one or more regulatory elements into the genome in such a position that it can increase expression of the cytokine(s). When the expression of multiple cytokines in the genome is needed to be increased in level, the genome may be modified to have one or more regulatory elements incorporated at the respective genomic sites of the cytokines.

In some embodiments, the one or more, and in some cases two or more, immunomodulatory cytokines that support T cell expansion, persistence, survival and function are utilized in compositions including CAR-expressing T cells, recombinant T cell receptor-expressing T cells, tumor antigen-specific T cells and/or virus-specific T cells.

### IV. Chimeric Antigen Receptors

Genetic engineering of human lymphocytes or other immune cells to express tumor-directed chimeric antigen receptors (CAR) can produce antitumor effector cells that bypass tumor immune escape mechanisms that are due to abnormalities in protein-antigen processing and presentation. Moreover, these transgenic receptors can be directed to tumor-associated antigens that are not protein-derived. In certain embodiments of the disclosure there are cytotoxic T lymphocytes (CTLs) that are modified to comprise a CAR that targets a tumor antigen, with GPC3 merely as an example.

In particular cases, T cells include a CAR receptor that is chimeric, non-natural and engineered at least in part by the hand of man. In particular cases, the engineered chimeric antigen receptor (CAR) has one, two, three, four, or more components, and in some embodiments the one or more components facilitate targeting or binding of the T lymphocyte to the tumor antigen-comprising cancer cell. In specific embodiments, the CAR comprises an antibody for the tumor antigen, part or all of a cytoplasmic signaling domain, and/or part or all of one or more co-stimulatory molecules, for example endodomains of co-stimulatory molecules. In specific embodiments, the antibody is a scFv.

In certain embodiments, a cytoplasmic signaling domain, such as those derived from the T cell receptor zeta-chain, is employed as at least part of the chimeric receptor in order to produce stimulatory signals for T lymphocyte proliferation and effector function following engagement of the chimeric receptor with the target antigen. Examples would include, but are not limited to, endodomains from co-stimulatory molecules such as CD28, CD27, 4-1BB, ICOS, OX40, a combination thereof, or the signaling components of cytokine receptors such as IL7 and IL15. In particular embodiments, co-stimulatory molecules are employed to enhance the activation, proliferation, and cytotoxicity of T cells produced by the GPC3 CAR after antigen engagement. In specific embodiments, the co-stimulatory molecules are CD28, 4-1BB, OX40, DAP10, DAP12, CD27, ICOS, for example.

The CAR may be first generation, second generation, or third generation (CAR in which signaling is provided by CD3zeta together with co-stimulation provided by CD28 and a tumor necrosis factor receptor (TNFR), such as 4-1BB or OX40), for example. The CAR may be specific for GPC3, and in some embodiments a GPC3-specific CAR-expressing cell may also express a second CAR targeting another antigen, including one or more CARs specific for CD19, CD20, CD22, Kappa or light chain, CD30, CD33, CD123, CD38, ROR1, ErbB2, ErbB3/4, EGFR vIII, carcinoembryonic antigen, EGP2, EGP40, mesothelin, TAG72, PSMA, NKG2D ligands, B7-H6, IL-13 receptor alpha2, IL-11 receptor R .alpha., MUC1, MUC16, CA9, GD2, GD3, HMW-MAA, CD171, Lewis Y, G250/CAIX, HLA-AI MAGE A1, HLA-A2 NY-ESO-1, PSC1, folate receptor-alpha, CD44v7/8, 8H9, NCAM, VEGF receptors, 5T4, Fetal AchR, NKG2D ligands, HER2, BCMA, or CD44v6, or other tumor-associated antigens or actionable mutations that are identified through genomic analysis and or differential expression studies of tumors, for example. In other cases, the CAR is bispecific for two non-identical antigens, including one referred to above in addition to being specific for GPC3, for example.

In particular cases the CAR is specific for GPC3, and in certain embodiments, the present disclosure provides chimeric T cells specific for GPC3 by joining an extracellular antigen-binding domain derived from a GPC3-specific antibody to cytoplasmic signaling domains derived from the T-cell receptor .zeta.-chain, optionally with the endodomains of the exemplary costimulatory molecules CD28 and OX40, for examples. This CAR is expressed in human cells, including human T cells, and the targeting of GPC3-positive cancers is encompassed herein.

Indicia of successful treatment could be, e.g., detectable reduction in the growth of a tumor (e.g., as seen by MRI or the like), or reduction in one or more symptoms of a cancer or other medical condition that expresses GPC3, including aberrantly expresses GPC3.

GPC3 may also be referred to as OCI-5, SDYS, GTR2-2, SGB, SGBS, SGBS1, MXR7, or DGSX, for example. An example of a GPC3 human nucleotide sequence is L47125 in GenBank® (with corresponding protein sequence in AAA98132 of GenBank®).

An example of a scFv for GPC3 is scFvGC33: Underlined: Leader; Bold scFv

(SEQ ID NO: 1)
MDWIWRILFLVGAATGAHSQVQLQQSGAELVRPGASVKLSCKASGYTFTD
YEMHWVKQTPVHGLKWIGALDPKTGDTAYSQKFKGKATLTADKSSSTAYM
ELRSLTSEDSAVYYCTRFYSYTYWGQGTLVTVSAGGGGSGGGGSGGGGSD
VVMTQTPLSLPVSLGDQASISCRSSQSLVHSNGNTYLHWYLQKPGQSPKL
LIYKVSNRFSGVPDRFSGSGSGTDFTLKISRVEAEDLGVYFCSQNTHVPP
TFGSGTKLEIK

A specific example of a GPC3-specific CAR is provided below in which IL21.IL15.GBBz construct: IL-21-Underlined; T2A-double underlined; IL-15-bold; GBBz (Glypican-3-specific chimeric antigen receptor with 4-1BB costimulatory endodomain)—bold and double underlined

(SEQ ID NO: 2)
MERIVICLMVIFLGTLVHKSSSQGQDRHMIRMRQLIDIVDQLKNYVNDLV
PEFLPAPEDVETNCEWSAFSCFQKAQLKSANTGNNERIINVSIKKLKRKP
PSTNAGRRQKHRLTCPSCDSYEKKPPKEFLERFKSLLKMIHHLSSRTHGS
EDSRAEGRGSLLTCGDVEENPGPMRISKPHLRSISIQCYLCLLLNSHFLT
EAGIHVFILGCFSAGLPKTEANWVNVISDLKKIEDLIQSMHIDATLYTES
DVHPSCKVTAMKCFLLELQVISLESGDASIHDTVENLIILANNSLSSNGN
VTESGCKECEELEEKNIKEFLQSFVHIVQMFINTSRAEGRGSLLTCGDVE
ENPGPMDWIWRILFLVGAATGAHSQVQLQQSGAELVRPGASVKLSCKASG
YTFTDYEMHWVKQTPVHGLKWIGALDPKTGDTAYSQKFKGKATLTADKSS
STAYMELRSLTSEDSAVYYCTRFYSYTYWGQGTLVTVSAGGGGSGGGGSG
GGGSDVVMTQTPLSLPVSLGDQASISCRSSQSLVHSNGNTYLHWYLQKPG
QSPKLLIYKVSNRFSGVPDRFSGSGSGTDFTLKISRVEAEDLGVYFCSQN
THVPPTFGSGTKLEIKEPKSCDKTHTCPPCPDPKFWVLVVVGGVLACYSL
LVTVAFIIKRGRKKLLYIFKQPFMRPVQTTQEEDGCSCRFPEEEEGGCEL
RVKFSRSADAPAYQQGQNQLYNELNLGRREEYDVLDKRRGRDPEMGGKPR
RKNPQEGLYNELQKDKMAEAYSEIGMKGERRRGKGHDGLYQGLSTATKDT
YDALHMQALPPR

### V. Introduction of Constructs into T Cells

Expression vectors that encode the tumor antigen-specific CARs and/or the cytokine(s) can be introduced into T cells as a DNA molecule or construct, where there may be at least one marker that will allow for selection of host cells that contain the construct(s). The constructs can be prepared in conventional ways, where the genes and regulatory regions may be isolated, as appropriate, ligated, cloned in an appropriate cloning host, analyzed by restriction or sequencing, or other convenient means. Particularly, using PCR, individual fragments including all or portions of a functional unit may be isolated, where one or more mutations may be introduced using “primer repair”, ligation, in vitro mutagenesis, etc., as appropriate. The construct(s) once completed and demonstrated to have the appropriate sequences may then be introduced into the CTL by any convenient means. The constructs may be integrated and packaged into non-replicating, defective viral genomes like Adenovirus, Adeno-associated virus (AAV), or Herpes simplex virus (HSV) or others, including retroviral vectors, for infection or transduction into cells. The constructs may include viral sequences for transfection, if desired. Alternatively, the construct may be introduced by fusion, electroporation, biolistics, transfection, lipofection, nanoparticles/nanocarriers, or the like. The host cells may be grown and expanded in culture before introduction of the construct(s), followed by the appropriate treatment for introduction of the construct(s) and integration of the construct(s). The cells are then expanded and screened by virtue of a marker present in the construct. Various markers that may be used successfully include hprt, neomycin resistance, thymidine kinase, hygromycin resistance, etc. The constructs may be introduced in situ, inside the human body into the target effector immune cells without ex vivo manipulation and or expansion.

In some instances, one may have a target site for homologous recombination, where it is desired that a construct be integrated at a particular locus. For example,) can knock-out an endogenous gene and replace it (at the same locus or elsewhere) with the gene encoded for by the construct using materials and methods as are known in the art for homologous recombination. For homologous recombination, one may use either OMEGA or O-vectors. See, for example, Thomas and Capecchi, Cell (1987) 51, 503-512; Mansour, et al., Nature (1988) 336, 348-352; and Joyner, et al., Nature (1989) 338, 153-156.

Vectors containing useful elements such as bacterial or yeast origins of replication, selectable and/or amplifiable markers, promoter/enhancer elements for expression in prokaryotes or eukaryotes, etc. that may be used to prepare stocks of construct DNAs and for carrying out transfections are well known in the art, and many are commercially available. Vectors that may be employed may be viral or non-viral. Examples of viral vectors include adenoviral, adeno-associated, lentiviral, or retroviral. Examples of non-viral vectors include plasmids, transposons, and so forth.

In some embodiments, the CAR and the cytokine(s) are delivered into the T cells on the same vector or on different vectors of the same or different type. When the CAR and the cytokine(s) are on the same vector, their expression construct may be separated by an IRES or 2A element. A variety of 2A sequence elements could be used to create linked- or co-expression of genes in the constructs provided in the present disclosure. For example, cleavage sequences could be used to co-express genes by linking open reading frames to form a single cistron. An exemplary cleavage sequence is the equine rhinitis A virus (E2A) or the F2A (Foot-and-mouth disease virus 2A) or a “2A-like” sequence (e.g., Thosea asigna virus 2A; T2A) or porcine teschovirus-1 (P2A). In specific embodiments, in a single vector the multiple 2A sequences are non-identical, although in alternative embodiments the same vector utilizes two or more of the same 2A sequences. Examples of 2A sequences are provided in US 2011/0065779 which is incorporated by reference herein in its entirety. In specific cases, two or more cytokines are delivered into the cell on the same vector and may be separated on a vector by a 2A or IRES element.

### VI. Administration of Cells

The exemplary T cells that have been modified with the construct(s) may be grown in culture under selective conditions and cells that are selected as having the construct may then be expanded and further analyzed, using, for example; the polymerase chain reaction for determining the presence of the construct in the host cells. Once the modified host cells have been identified, they may then be used as planned, e.g. expanded in culture or introduced into a host organism.

Depending upon the nature of the cells, the cells may be introduced into a host organism, e.g., a mammal, in a wide variety of ways. The cells may be introduced at the site of the tumor, in specific embodiments, although in alternative embodiments the cells hone to the cancer or are modified to hone to the cancer. The number of cells that are employed will depend upon a number of circumstances, the purpose for the introduction, the lifetime of the cells, the protocol to be used, for example, the number of administrations, the ability of the cells to multiply, the stability of the recombinant construct, and the like. The cells may be applied as a dispersion, generally being injected at or near the site of interest. The cells may be in a physiologically-acceptable medium.

The DNA introduction need not result in integration in every case. In some situations, transient maintenance of the DNA introduced may be sufficient. In this way, one could have a short term effect, where cells could be introduced into the host and then turned on after a predetermined time, for example, after the cells have been able to home to a particular site.

The cells may be administered as desired. Depending upon the response desired, the manner of administration, the life of the cells, the number of cells present, various protocols may be employed. The number of administrations will depend upon the factors described above at least in part.

It should be appreciated that the system is subject to many variables, such as the cellular response to the ligand, the efficiency of expression and, as appropriate, the level of secretion, the activity of the expression product, the particular need of the patient, which may vary with time and circumstances, the rate of loss of the cellular activity as a result of loss of cells or expression activity of individual cells, and the like. Therefore, it is expected that for each individual patient, even if there were universal cells which could be administered to the population at large, each patient would be monitored for the proper dosage for the individual, and such practices of monitoring a patient are routine in the art. One example of a dose of cells is in a range of 10⁴/kg to 10⁹/kg, including 10⁴-10⁸, 10⁴-10⁷, 10⁴-10⁶, 10⁴-10⁵, 10⁵-10⁹, 10⁵-10⁸, 10⁵-10⁷, 10⁵-10⁶, 10⁶-10⁹, 10⁶-10⁸, 10⁶-10⁷, 10⁷-10⁹, or 10⁷-10⁸.

In particular embodiments, nanoparticles are utilized as a vector. The nanoparticles carry nucleic acid sequences that can be inserted into the host DNA by enzymes (i.e. transposases) (Smith, T. T., Stephan, S. B., et al., Nature Nanotechnology, In situ programming of leukaemia-specific T cells using synthetic DNA nanocarriers, 2017). As shown therein, DNA-carrying nanoparticles can efficiently introduce CAR genes into nuclei of T cells. Thus, in specific embodiments the cells are in situ engineered. The vector (viral or non-viral) may be introduced into the individual (for example, injected) and the T cells are engineered inside the human body Nanoparticles, AAVs, and lentiviruses may be employed for in situ engineering, for example. In a specific case, the vector is injected into the blood of patients and the T cells are engineered inside the body.

### VII. Examples of Methods of Treatment

Adoptive transfer of T lymphocytes (including CAR-expressing, recombinant TCR-expressing, virus-specific, or tumor antigen-specific cells) represents a useful therapy for patients with malignancies. Here the applicability of this strategy is extended to a broad array of solid tumors by targeting the GPC3 antigen. Particular aspects of the disclosure include methods of treating GPC3-expressing cancers.

By way of illustration, individuals with cancer or at risk for cancer (such as having one or more risk factors) or suspected of having cancer may be treated as follows. Modified T cells as described herein may be administered to the individual and retained for extended periods of time. The individual may receive one or more administrations of the cells, and the administrations may or may not occur in conjunction with one or more other cancer therapies. In some embodiments, the genetically modified cells are encapsulated to inhibit immune recognition and placed at the site of the tumor.

In particular cases, an individual is provided with therapeutic CTLs modified to comprise a CAR specific for a tumor antigen, such as GPC3, and one or more interleukins in addition to other types of therapeutic cells. The cells may be delivered at the same time or at different times. The cells may be delivered in the same or separate formulations. The cells may be provided to the individual in separate delivery routes. The cells may be delivered by injection at a tumor site or intravenously or orally, for example. The cells may be delivered systemically or locally. Routine delivery routes for such compositions are known in the art.

The GPC3-expressing cancers may be of any kind, including at least liver, testicular, lung, ovarian, head and neck cancer, mesothelioma, breast, glioblastoma, kidney, brain, skin, colon, prostate, pancreatic, cervical, thyroid, spleen, or bone cancer, for example. In particular cases, the cancer is hepatoblastoma, hepatocellular carcinoma, malignant rhabdoid tumors, yok sac tumors, undifferentiated sarcoma of the liver, liposarcoma, Wilm's tumor, or choriocarcinoma.

In various embodiments tumor antigen-targeting CAR constructs, nucleic acid sequences, vectors, host cells, as contemplated herein and/or pharmaceutical compositions comprising the same are used for the prevention, treatment or amelioration of a cancerous disease, such as a tumorous disease. In particular embodiments, the pharmaceutical composition of the present disclosure may be particularly useful in preventing, ameliorating and/or treating cancer, including cancer that express GPC3 and that may or may not be solid tumors, for example.

As used herein “treatment” or “treating,” includes any beneficial or desirable effect on the symptoms or pathology of a disease or pathological condition and may include even minimal reductions in one or more measurable markers of the disease or condition being treated, e.g., cancer. Treatment can involve optionally either the reduction or amelioration of symptoms of the disease or condition, or the delaying of the progression of the disease or condition. “Treatment” does not necessarily indicate complete eradication or cure of the disease or condition, or associated symptoms thereof.

As used herein, “prevent,” and similar words such as “prevented,” “preventing” etc., indicate an approach for preventing, inhibiting, or reducing the likelihood of the occurrence or recurrence of, a disease or condition, e.g., cancer. It also refers to delaying the onset or recurrence of a disease or condition or delaying the occurrence or recurrence of the symptoms of a disease or condition. As used herein, “prevention” and similar words also includes reducing the intensity, effect, symptoms and/or burden of a disease or condition prior to onset or recurrence of the disease or condition.

In particular embodiments, the present disclosure contemplates, in part, GPC3 CAR-expressing cells, GPC3 CAR constructs, GPC3 CAR nucleic acid molecules and GPC3 CAR vectors that are modified to provide one or more interleukins and can administered either alone or in any combination using standard vectors and/or gene delivery systems, and in at least some aspects, together with a pharmaceutically acceptable carrier or excipient. In certain embodiments, subsequent to administration, the CAR and/or IL nucleic acid molecules or vectors may be stably integrated into the genome of the subject.

In specific embodiments, viral vectors may be used that are specific for certain cells or tissues and persist in said cells. Suitable pharmaceutical carriers and excipients are well known in the art. The compositions prepared according to the disclosure can be used for the prevention or treatment or delaying the above identified diseases.

Furthermore, the disclosure relates to a method for the prevention, treatment or amelioration of a tumorous disease comprising the step of administering to a subject in the need thereof an effective amount of cells that express a GPC3-targeting CAR, a nucleic acid sequence, a vector, as contemplated herein and/or produced by a process as contemplated herein.

Possible indications for administration of the composition(s) of the exemplary GPC3 CAR cells are cancerous diseases, including tumorous diseases, including hepatocellular carcinoma, a hepatoblastoma, an embryonal sarcoma, a rhabdoid tumor, a Wilm's tumor, yolk sac tumor, choriocarcinoma, a squamous cell carcinoma of the lung, a liposarcoma, a breast carcinoma, a head and neck squamous cell carcinoma (HNSCC), or mesothelioma, for example. Exemplary indications for administration of the composition(s) of tumor antigen-specific CAR cells are cancerous diseases, including any malignancies that express GPC3. The administration of the composition(s) of the disclosure is useful for all stages and types of cancer, including for minimal residual disease, early cancer, advanced cancer, and/or metastatic cancer and/or refractory cancer, for example.

The disclosure further encompasses co-administration protocols with other compounds, e.g. bispecific antibody constructs, targeted toxins or other compounds, which act via immune cells. The clinical regimen for co-administration of the inventive compound(s) may encompass co-administration at the same time, before or after the administration of the other component. Particular combination therapies include chemotherapy, radiation, surgery, hormone therapy, or other types of immunotherapy.

Embodiments relate to a kit comprising a tumor antigen-specific CAR construct as defined herein, a nucleic acid sequence as defined herein, a vector as defined herein and/or a host as defined herein. It is also contemplated that the kit of this disclosure comprises a pharmaceutical composition as described herein above, either alone or in combination with further medicaments to be administered to an individual in need of medical treatment or intervention.

### VIII. Combination Therapy

In certain embodiments of the disclosure, methods of the present disclosure for clinical aspects are combined with other agents effective in the treatment of hyperproliferative disease, such as anti-cancer agents. An “anti-cancer” agent is capable of negatively affecting cancer in a subject, for example, by killing cancer cells, inducing apoptosis in cancer cells, reducing the growth rate of cancer cells, reducing the incidence or number of metastases, reducing tumor size, inhibiting tumor growth, reducing the blood supply to a tumor or cancer cells, promoting an immune response against cancer cells or a tumor, preventing or inhibiting the progression of cancer, or increasing the lifespan of a subject with cancer. More generally, these other compositions would be provided in a combined amount effective to kill or inhibit proliferation of the cell. This process may involve contacting the cancer cells with the expression construct and the agent(s) or multiple factor(s) at the same time. This may be achieved by contacting the cell with a single composition or pharmacological formulation that includes both agents, or by contacting the cell with two distinct compositions or formulations, at the same time, wherein one composition includes the expression construct and the other includes the second agent(s).

Tumor cell resistance to chemotherapy and radiotherapy agents represents a major problem in clinical oncology. One goal of current cancer research is to find ways to improve the efficacy of chemo- and radiotherapy by combining it with gene therapy. For example, the herpes simplex virus-thymidine kinase (HSV-tK) gene, when delivered to brain tumors by a retroviral vector system, successfully induced susceptibility to the antiviral agent ganciclovir (Culver, et al., 1992). In the context of the present disclosure, it is contemplated that cell therapy could be used similarly in conjunction with chemotherapeutic, radiotherapeutic, or immunotherapeutic intervention, in addition to other pro-apoptotic or cell cycle regulating agents.

Alternatively, the present inventive therapy may precede or follow the other agent(s) treatment by intervals ranging from minutes to weeks. In embodiments where the other agent and present disclosure are applied separately to the individual, one would generally ensure that a significant period of time did not expire between the time of each delivery, such that the agent and inventive therapy would still be able to exert an advantageously combined effect on the cell. In such instances, it is contemplated that one may contact the cell with both modalities within about 12-24 h of each other and, more preferably, within about 6-12 h of each other. In some situations, it may be desirable to extend the time period for treatment significantly, however, where several d (2, 3, 4, 5, 6 or 7) to several wk (1, 2, 3, 4, 5, 6, 7 or 8) lapse between the respective administrations.

It is expected that the treatment cycles would be repeated as necessary. It also is contemplated that various standard therapies, as well as surgical intervention, may be applied in combination with the inventive cell therapy.

A. Chemotherapy

Cancer therapies also include a variety of combination therapies with both chemical and radiation-based treatments. Combination chemotherapies include, for example, abraxane, altretamine, docetaxel, herceptin, methotrexate, novantrone, zoladex, cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, taxol, gemcitabien, navelbine, farnesyl-protein tansferase inhibitors, transplatinum, 5-fluorouracil, vincristin, vinblastin and methotrexate, or any analog or derivative variant of the foregoing and also combinations thereof.

In specific embodiments, chemotherapy for the individual is employed in conjunction with the disclosure, for example before, during and/or after administration of the disclosure.

B. Radiotherapy

Other factors that cause DNA damage and have been used extensively include what are commonly known as .gamma.-rays, X-rays, and/or the directed delivery of radioisotopes to tumor cells. Other forms of DNA damaging factors are also contemplated such as microwaves and UV-irradiation. It is most likely that all of these factors effect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

The terms “contacted” and “exposed,” when applied to a cell, are used herein to describe the process by which a therapeutic construct and a chemotherapeutic or radiotherapeutic agent are delivered to a target cell or are placed in direct juxtaposition with the target cell. To achieve cell killing or stasis, both agents are delivered to a cell in a combined amount effective to kill the cell or prevent it from dividing.

C. Immunotherapy

Immunotherapeutics generally rely on the use of immune effector cells and molecules to target and destroy cancer cells. The immune effector may be, for example, an antibody specific for some marker on the surface of a tumor cell. The antibody alone may serve as an effector of therapy or it may recruit other cells to actually effect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells.

Immunotherapy other than the inventive therapy described herein could thus be used as part of a combined therapy, in conjunction with the present cell therapy. The general approach for combined therapy is discussed below. Generally, the tumor cell must bear some marker that is amenable to targeting, i.e., is not present on the majority of other cells. Many tumor markers exist and any of these may be suitable for targeting in the context of the present disclosure. Common tumor markers include carcinoembryonic antigen, prostate specific antigen, urinary tumor associated antigen, fetal antigen, tyrosinase (p97), gp68, TAG-72, HMFG, Sialyl Lewis Antigen, MucA, MucB, PLAP, estrogen receptor, laminin receptor, erb B and p155.

D. Genes

In yet another embodiment, the secondary treatment is a gene therapy in which a therapeutic polynucleotide is administered before, after, or at the same time as the present disclosure clinical embodiments. A variety of expression products are encompassed within the disclosure, including inducers of cellular proliferation, inhibitors of cellular proliferation, or regulators of programmed cell death.

E. Surgery

Approximately 60% of persons with cancer will undergo surgery of some type, which includes preventative, diagnostic or staging, curative and palliative surgery. Curative surgery is a cancer treatment that may be used in conjunction with other therapies, such as the treatment of the present disclosure, chemotherapy, radiotherapy, hormonal therapy, gene therapy, immunotherapy and/or alternative therapies.

Curative surgery includes resection in which all or part of cancerous tissue is physically removed, excised, and/or destroyed. Tumor resection refers to physical removal of at least part of a tumor. In addition to tumor resection, treatment by surgery includes laser surgery, cryosurgery, electrosurgery, and microscopically-controlled surgery (Mohs' surgery). It is further contemplated that the present disclosure may be used in conjunction with removal of superficial cancers, precancers, or incidental amounts of normal tissue.

Upon excision of part of all of cancerous cells, tissue, or tumor, a cavity may be formed in the body. Treatment may be accomplished by perfusion, direct injection or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

F. Other Agents

It is contemplated that other agents may be used in combination with the present disclosure to improve the therapeutic efficacy of treatment. These additional agents include immunomodulatory agents, agents that affect the upregulation of cell surface receptors and GAP junctions, cytostatic and differentiation agents, inhibitors of cell adhesion, or agents that increase the sensitivity of the hyperproliferative cells to apoptotic inducers. Immunomodulatory agents include tumor necrosis factor; interferon alpha, beta, and gamma; IL-2 and other cytokines; F42K and other cytokine analogs; or MIP-1, MIP-1beta, MCP-1, RANTES, and other chemokines. It is further contemplated that the upregulation of cell surface receptors or their ligands such as Fas/Fas ligand, DR4 or DRS/TRAIL would potentiate the apoptotic inducing abilities of the present disclosure by establishment of an autocrine or paracrine effect on hyperproliferative cells. Increases intercellular signaling by elevating the number of GAP junctions would increase the anti-hyperproliferative effects on the neighboring hyperproliferative cell population. In other embodiments, cytostatic or differentiation agents can be used in combination with the present disclosure to improve the anti-hyperproliferative efficacy of the treatments. Inhibitors of cell adhesion are contemplated to improve the efficacy of the present disclosure. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with the present disclosure to improve the treatment efficacy.

### EXAMPLES

The following examples are included to demonstrate preferred embodiments of the invention. It should be appreciated by those of skill in the art that the techniques disclosed in the examples that follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Glypican-3-Specific CAR T Cells Co-Expressing Interleukin-15 and -21 have Superior Expansion and Antitumor Activity Against Hepatocellular Carcinoma

Hepatocellular carcinoma (HCC) is the fourth most common cause of cancer-related death in the world (1). Lack of curative therapies for unresectable and/or metastatic disease, which occurs in the majority of newly diagnosed cases and results in dismal prognoses (1). Chimeric antigen receptor (CAR)-expressing T cells have shown breakthrough clinical successes for the treatment of CD19-positive hematological malignancies (2-6) In contrast, CAR T cells have demonstrated only modest anti-tumor activity in patients with solid tumors including neuroblastoma, sarcomas, and HCC, in part due to their limited expansion and persistence (7-12). As the overall therapeutic efficacy of CAR T cells strongly correlates with their expansion and persistence in patients with CD19-positive malignancies (2,4), translational approaches to enhance these properties may improve the antitumor efficacy of CAR T therapy in patients with HCC.

Human interleukin-15 (IL-15) and IL-21 are required for optimal T cell activation, expansion, differentiation, and function (13,14). These cytokines are notably absent in the HCC microenvironment, depriving T cells of survival signals upon tumor cell engagement (13,14). In preclinical models of CD19+ malignancies, neuroblastoma or gliomas, CAR T cells co-expressing either IL-15 or IL-21 controlled tumors significantly better than CAR T cells alone (15-18). Additionally, IL-15 and IL-21 have been shown to synergistically promote antigen-dependent T cell expansion and cytolytic function (19,20). However, whether IL-15 or IL-21 enhance the antitumor effector function of CAR T cells against HCC remains to be seen. Further, it is unknown whether the combination of both cytokines could further improve the antitumor properties of CAR T cells. In a recent study, there was evaluation of the in vitro and in vivo activity of T cells expressing CARs targeting glypican-3 (GPC3), an antigen expressed in over 70% of HCC cases but not in non-malignant tissues (21-27). The GPC3-CAR containing a 4-1BB costimulatory endodomain—‘GBBz’—was selected for further characterization, as this receptor induced favorable T_{H}1-polarized effector cytokine release upon tumor cell engagement and produced superior expansion and antitumor activity (27). In the present example, to determine the impact of IL-15 and IL-21 on T cell survival, persistence, and anti-tumor activity in preclinical models of HCC, IL-15, IL-21 or both were co-expressed with the GBBz GPC3-CAR in T cells.

It is demonstrated herein that GBBz GPC3-CAR T cells co-expressing IL-15 and/or IL-21 specifically and effectively kill GPC3-positive tumor cells including HCC in an antigen-dependent manner. The results also indicate that constitutive transgenic expression of both cytokines together enriches for less differentiated T cells, which are better protected from apoptosis during repeated exposures to tumor cells. Combined IL-15/IL-21 expression maintains the expression of T cell factor-1 (TCF-1), a transcription factor critical for T cell development and survival. Finally, GPC3-CAR T cells co-expressing both IL-15 and IL-21 exhibit the most robust peak expansion and sustained persistence in vivo and that these properties translate to superior tumor control in and survival of HCC tumor-bearing mice.

In the present example, GBBz-based GPC3-CAR T cells co-expressing IL-15 and/or IL-21 specifically and effectively kill GPC3-positive tumor cells in an antigen-dependent manner. The results also indicate that constitutive transgenic expression of both cytokines together enriches for less differentiated T cells that are then protected from apoptosis during repeated exposures to tumor cells. Combined IL-15/IL-21 expression maintains the expression of TCF-1, a transcription factor critical for T cell development and survival. Finally, GPC3-CAR T cells co-expressing both IL-15 and IL-21 exhibit the most robust peak expansion and sustained persistence in vivo and that these properties translate into superior tumor control in and survival of HCC tumor-bearing mice. These results have provided strong rationale for including the IL-15/IL-21 co-expressing with a CAR in patients with HCC and other solid cancers.

Examples of Materials and Methods

Cell Lines

The HCC cell line Hep3B, rhabdoid tumor cell line G401, lung carcinoma cell line A549 and human embryonic kidney cell line 293T were obtained from the American Type Culture Collection (Manassas, Va.). The HCC cell line Huh-7 was a kind gift from Dr. Xiao-Tong Song (Baylor College of Medicine, Houston, Tex.) and its identity was confirmed at the Characterized Cell Line Core Facility at MD Anderson Cancer Center (Houston, Tex.). A549-GPC3 cells were generated by transducing A549 cells with a retroviral vector encoding GPC3; Huh-7 firefly luciferase (Ffluc) cells were similarly generated using an eGFP.Ffluc construct (Li et al., 2017). Each cryopreserved cell line vial was subject to a maximum of four weeks subculture after recovery. Cell lines Huh-7, Hep3B, G401, and A549 were cultured in Dulbecco's Modified Eagle Medium (DMEM) supplemented with 10% fetal bovine serum and 2 mM GlutaMAX. 293T cells were cultured in Iscove's Modified Dulbecco's Medium supplemented with 10% fetal bovine serum and 2 mM GlutaMAX. Cells were cultured at 37° C. in humidified air containing 5% CO2.

Generation of Retroviral Constructs

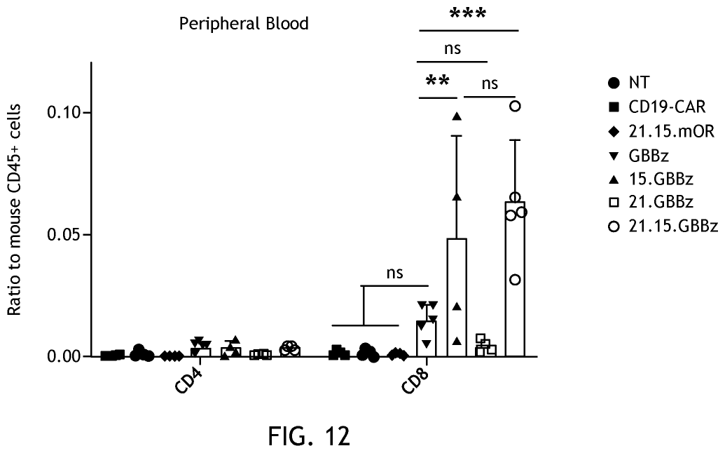

A codon optimized minigene encoding cytokines IL-21, IL-15, and the GPC3-CAR ‘GBBz’ (Li et al., 2017) linked with a T2A sequence and flanked by NcoI and MluI restriction enzyme sites was synthesized by GeneArt® (Thermo Fisher Scientific, Waltham, Mass.). This fragment was subcloned in frame into the pSFG retroviral vector using NcoI and MluI restriction enzymes yielding the 21.15.GBBz retroviral construct (FIG. 12). 21.GBBz was generated from 21.15.GBBz by PCR amplifying genes encoding IL-21

(F: ATCCTCTAGACTGCCATGGAACGGATC (SEQ ID NO: 3)/
R: CGTCCCTCGGCTCTAGAATCTTCG; (SEQ ID NO: 4))
and
GBBz

(F: TAGAGCCGAGGGACGGGGCT (SEQ ID NO:5)/R: ATGATGACGCGTTAATCATCTGGGGGG; (SEQ ID NO:6)), followed by In-Fusion® cloning (Takara Bio, Mountain View, Calif.) to insert fragments into the pSFG retroviral vector backbone (FIG. 12). 15.GBBz was similarly generated by amplifying the gene fragment encoding both IL-15 and GBBz (F: ATCCTCTAGACTGCCATGAGAATCAGCAAGCCCC (SEQ ID NO:7)/R: ATGATGACGCGTTAATCATCTGGGGGG (SEQ ID NO:8)) and inserting it into the pSFG plasmid backbone by In-Fusion® cloning (FIG. 12). The 21.15 construct was produced by PCR amplifying the fragment encoding both IL-21 and IL-15 from 21.15.GBBz (F: ATCCTCTAGACTGCCATGGAACGGATC (SEQ ID NO:9)/R: CAGTGCGGCCGCTCAGGCCCTGCTGGTGTT; (SEQ ID NO:10)) and in the process generating NcoI and NotI restriction enzyme sites in the introduced 5′ and 3′ overhangs. This fragment was digested with NcoI and NotI and cloned into a pSFG plasmid bearing the orange monomeric derivative of DsRed fluorescent protein (Iwahori et al., 2015). Sequencing was performed following each cloning step (Epoch Life Science, Sugar Land, Tex.). A CD19-specific CAR (FMC63 scFv, CD28 and 4-1BB costimulatory domains, CD3ζ signaling endodomain) was used as negative control (Ramos et al., 2018).

Retrovirus Production and Transduction of Primary T Cells and Cell Lines

Retroviral packaging and transduction were performed as described previously (Li et al., 2017).

Cytotoxicity Assay

Cytotoxicity of GPC3-CAR T cells was assessed as described previously (Li et al., 2017) using a standard four-hour chromium 51 (⁵¹Cr) release assay. Briefly, target cells were labelled with ⁵¹Cr for 1 hour followed by incubation with effector cells for four hours at 37° C. using multiple effector-to-target ratios. Cell culture supernatants were collected, and radioactivity was measured in a gamma counter (PerkinElmer, Waltham, Mass.).

Measurement of Cytokines and Chemokines

Enzyme-linked immunosorbent assays (ELISAs) were performed to measure transgenic expression of IL-15 and IL-21 using the Human IL-15/IL-21 ELISA MAX™ Deluxe kit (Biolegend, San Diego, Calif.) according to the manufacturer's instructions. Briefly, 0.5×10⁶ resting CAR T cells were cultured in the presence or absence of Huh-7 cells at 1:1 ratio. Cell culture supernatants were collected at 72 hours, centrifuged, and frozen until the time of assay. Cytokine concentrations were calculated using a best fit line of optical density and concentration generated with pre-calibrated protein standards. A correlation coefficient (R²) >0.9 compared to pre-calibrated standards was required.

Multiplex cytokine/chemokine immunoassays were performed as described previously (Li et al., 2017) to measure CAR T cells' effector cytokine production. Supernatants were assayed using the MILLIPLEX MAP human cytokine/chemokine magnetic bead kit (EMD Millipore, Billerica, Mass.) according to the manufacturer's instructions.

Flow Cytometry

GPC3-CAR expression was detected using the anti-F(ab)₂ Alexa Fluor® 647-conjugated antibody (Jackson ImmunoResearch) and anti-goat IgG₁ isotype control (Jackson ImmunoResearch, West Grove, Pa.). The following antibodies were used for T cell phenotyping analyses: anti-CD4-APC/Fire 750 (BioLegend), anti-CD8-V500 (BD Biosciences, San Jose, Calif.), anti-CD45RO-PE/Cy7 (BioLegend), anti-CD62L-AF488 (BioLegend), anti-CD19-PerCP/Cy5.5 (CCR1; BioLegend), anti-CD3-PE (BD Biosciences), anti-CD279-PerCP/Cy5.5 (PD-1; BioLegend), anti-CD223-PE/Cy7 (LAG-3; BioLegend) and anti-CD366-BV421 (TIM-3; BD Biosciences). Anti-bovine IgG antibody (Sigma-Aldrich, St. Louis, Mo.) was used to block non-specific binding of other murine antibodies following CAR staining. Flow cytometry assessment was performed on either an LSR-II (BD Biosciences) or iQue Screener PLUS (Intellicyt Corporation, Albuquerque, N. Mex.). Results were analyzed using FlowJo software (FlowJo, Ashland, Oreg.). To detect intracellular TCF-1 expression, CAR T cells were first stained for surface expression of CAR, CD4, and CD8 as above, followed by staining with anti-TCF1-PE (TCF7, BioLegend) used in conjunction with the True-Nuclear™ Transcription Factor Buffer Set (BioLegend) according to the manufacturer's instructions. To inhibit STAT3 and STAT5, CAR T cells were cultured in media containing inhibitors S3I-201 (2504) and Pimozide (504), respectively, or DMSO (control) for 24 hours prior to staining for TCF-1.

Repeat Stimulation Stress Test

In vitro expansion and persistence were assessed by repeatedly co-culturing CAR T cells with fresh Huh-7 tumor cells every 3-4 days at a 1:1 ratio. At the end of each co-culture interval, CAR T cell counts were measured by flow cytometry staining for the CAR and using CountBright™ beads (ThermoFischer. Waltham, Mass.) Annexin V staining was performed to assess CAR T cell viability two days after each stimulation using the ApoScreen® Annexin V Apoptosis Kit (SouthernBiotech, Birmingham, Ala.) according to the manufacturer's instructions.

RNA Extraction and Sequencing

CAR T cells were sorted using a Sony SH800Z instrument (Sony Biotechnology, San Jose, Calif.) and expanded for one week in complete RPMI supplemented with 100 units/ml penicillin, 100 μg/ml streptomycin and 0.25 μg/ml amphotericin B (Thermofisher Scientific). Sorterd CAR T cells were co-cultured with tumor cells at a 2:1 (E:T) ratio for three days. To confirm complete tumor cell lysis prior to RNA extraction, co-cultured cells were analyzed by flow cytometry. RNA was extracted using the RNeasy Mini Kit (Qiagen, Germantown, Md.) as per the manufacturer's protocol. RNA sequencing was performed at the Genomic and RNA Profiling Core at Baylor College of Medicine (BCM) using the nCounter Analysis System (NanoString Technologies, Seattle, Wash.) and the pre-defined nCounter Human Immunology V2 panel. Gene expression data was normalized and analyzed using nSolver software (NanoString Technologies). Benjamini-Hochberg correction was used for multiple comparisons.

In Vivo Experiments:

All mice used in this study were maintained at the Small Animal Core Facility of Texas Children's Hospital and handled under protocols approved by BCM's Institutional Biosafety Committee and Institutional Animal Care and Use Committee.

In vivo anti-tumor activity of infused CAR T cells and mouse survival were evaluated in murine HCC xenograft models as described previously (Li et al., 2017) with modifications. Briefly, 12-week-old female NOD.Cg-Prkdc^{scid} Il2rg^{tm1Wjl}/SzJ (NSG, The Jackson Laboratory, Bar Harbor, Me.) mice were injected intraperitoneally with 2×10⁶ Ffluc+Huh-7 cells or 5×10⁶ Ffluc+G401 cells followed by 0.5×10⁶ or 2×10⁶ CAR T cells intravenously one or two weeks later, as indicated. Mice were assessed daily and tumor bioluminescence was measured using the IVIS Lumina III imaging system (PerkinElmer, Waltham, Mass.).

To evaluate CAR T cell in vivo proliferation and persistence, mice were injected with 2×10⁶ Huh-7 cells followed two weeks later by 2×10⁶ CAR T cells co-expressing an optimized Ffluc (Rabinovich et al., 2008). Mice were imaged every other day following CAR T cell injection to monitor expansion. Blood and spleens were collected on days 15 and 18, respectively, and evaluated for the presence of CAR T cells by flow cytometry. Cells were stained for mouse CD45 using anti-mouse CD45-PE or PerCP/Cy5.5 (BioLegend, Cat. #103106, Cat. #103132), human CD4, human CD8, and the GPC3-CAR as described above. IL-15 and IL-21 levels in the plasma were measured using the MILLIPLEX MAP human cytokine/chemokine magnetic bead kit (EMD Millipore).

Statistical Analyses

Data were summarized using descriptive statistics. ANOVAs followed by pairwise comparisons between groups were carried out, taking into account matched donors if applicable. Response variables were log-transformed if necessary to achieve normality. Analysis was performed using SAS version 9.4. P values <0.05 were considered statistically significant.

T Cells Effectively Co-Express IL-21 and/or IL-15 with an Optimized GPC3-CAR from a Single Retroviral Construct and Production of these Cytokines Increases Upon CAR T Cell Activation.

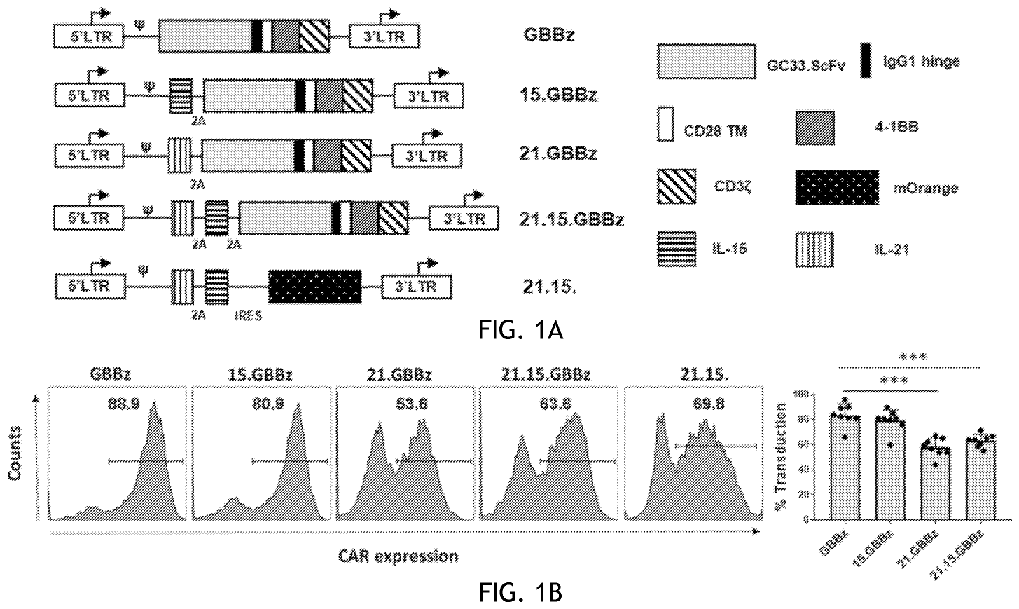

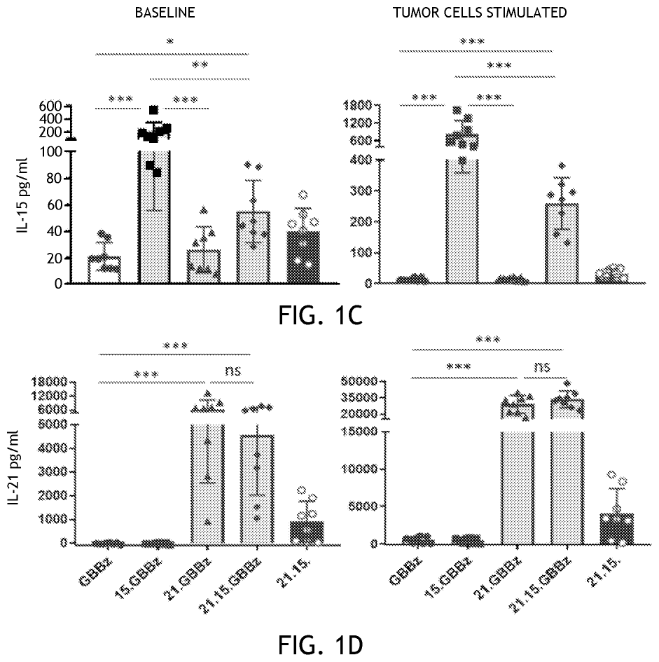

A set of CAR constructs were generated based on optimization of GBBz GPC3-CAR (27) with additional sequence(s) for human IL-21 and/or IL-15 (FIG. 1A) using the clinically validated Moloney murine leukemia virus-derived SFG retroviral vector backbone. After transduction, all constructs were stably expressed by human peripheral blood T cells, with constructs containing IL-21 (21.GBBz and 21.15.GBBz) demonstrating slightly lower overall transduction efficiency compared to the GBBz construct (p<0.001; FIG. 1B).

To measure IL-15 and IL-21 production, supernatants were collected from GPC3-CAR T cells cultured with and without GPC3-positive tumor cells and evaluated by ELISA. The inventor confirmed that IL-15 and IL-21 were indeed secreted by CAR T cells engineered to express the corresponding genes at baseline (FIGS. 1C and 1D). Following CAR stimulation by GPC3-positive HCC cells, IL-15 and IL-21 production increased significantly from T cells co-expressing the corresponding transgenes (p<0.001), and IL-21 concentrations remained significantly higher than IL-15 levels (p<0.001). While T cells expressing 15.GBBz produced significantly more IL-15 than 21.15.GBBz T cells at baseline (p<0.01) and after stimulation (p<0.001), IL-21 production levels did not differ between 21.GBBz and 21.15.GBBz groups in either condition.

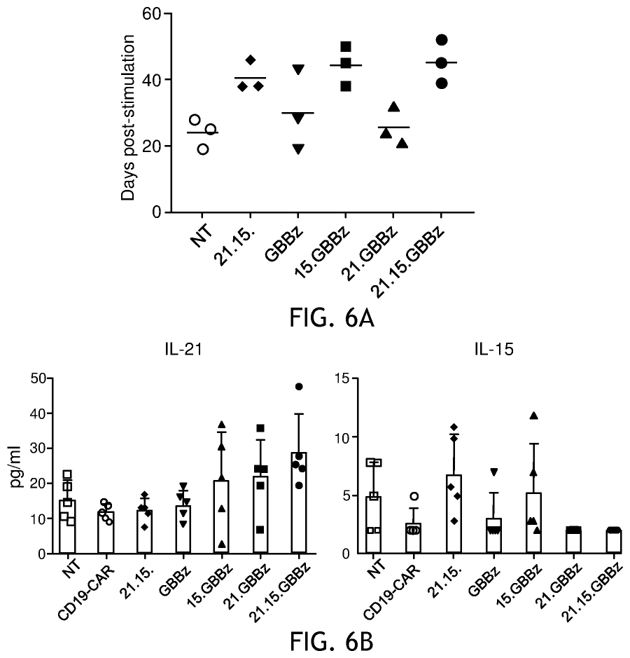

Given that IL-15 and/or IL-21 could potentially induce antigen-independent T cell proliferation, the ability of each T cell group to maintain autonomous growth was evaluated. While all groups underwent an initial burst of proliferation after transduction, in the absence of antigen stimulation, no viable CAR T cells remained in any group after 50 days (FIG. 6A).

In summary, transduced T cells stably express GPC3-CAR constructs and produce significant quantities of one or both cytokines, as appropriate, without evidence of antigen-independent autonomous growth.

Co-Expression of IL-15 and/or IL-21 does not Impact Short-Term Cytotoxic Function of GPC3-CAR T Cells but Alters their Cytokine Production Profile.

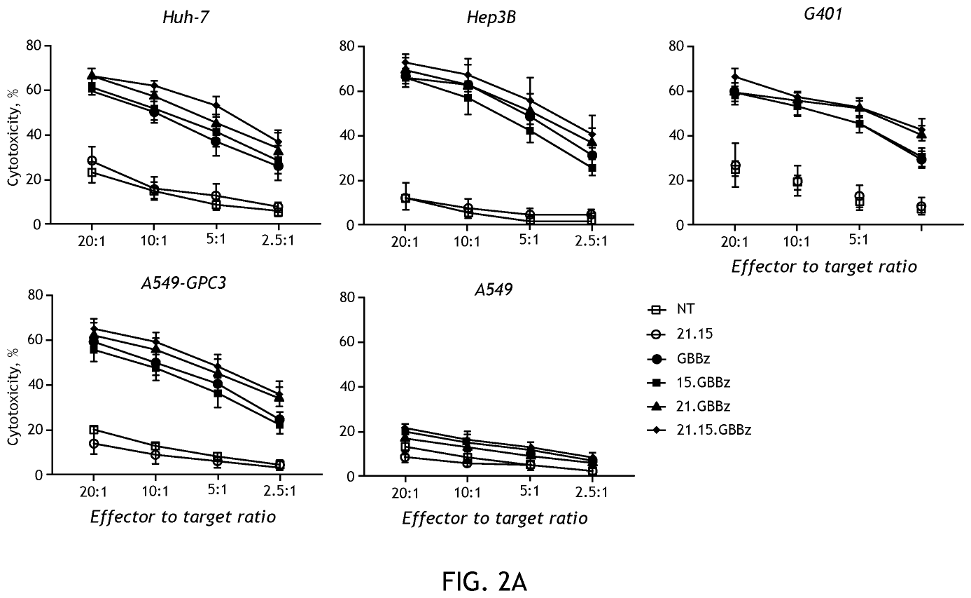

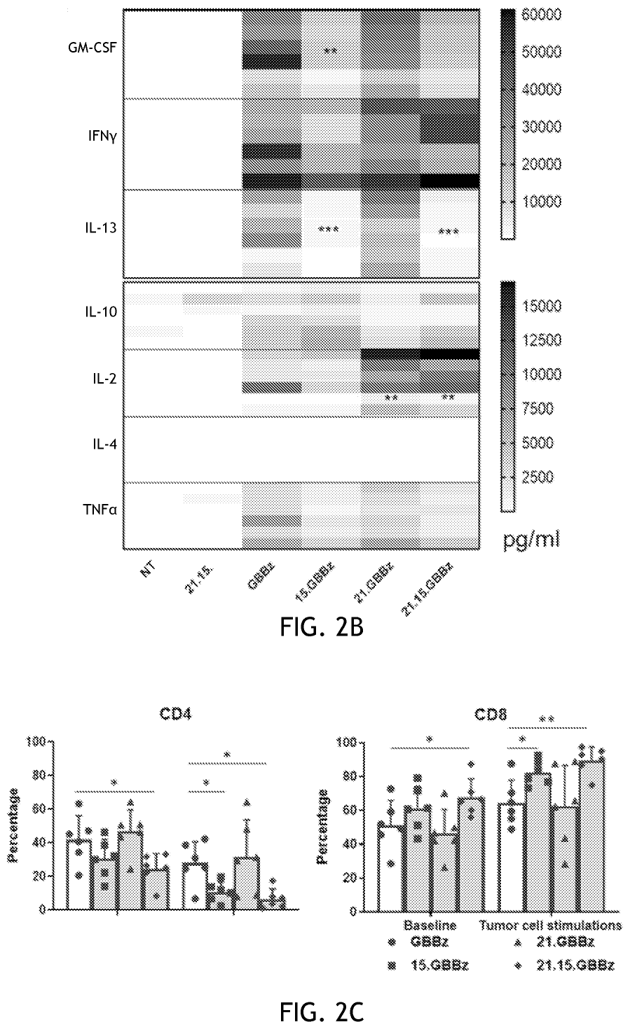

It was next explored whether IL-21 and/or IL-15 co-expression impacts the efficacy and/or specificity of GPC3-CAR-mediated tumor cell killing using a chromium-51 release assay (27). T cells expressing any of the four GPC3-CAR constructs specifically and effectively lysed GPC3-positive tumor cells (Huh-7, Hep3B, G401, A549-GPC3) in an antigen-dependent manner regardless of IL-21/IL-15 co-expression (FIG. 2A).

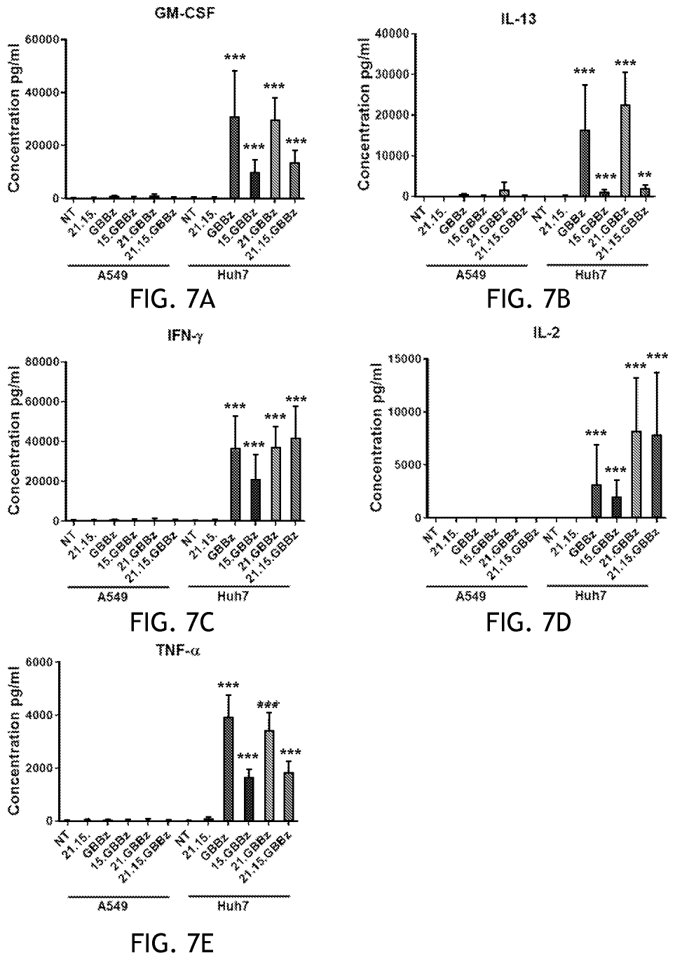

To evaluate an additional measure of T cell activation, we determined the T_{H}1 and T_{H}2 effector cytokine production profiles of GPC3-CAR T cells following co-culture with GPC3-positive or -negative target cells. CAR engagement by GPC3-positive Huh-7 cells specifically induced cytokine production by GPC3-CAR T cells but not by control groups (FIG. 2B; FIG. 7). IL-15 co-expression caused a significant decrease in IL-13 production compared to T cells expressing constructs without IL-15 (p<0.001; FIG. 2B). A similar decline was observed for GM-CSF in 15.GBBz but not in 21.15.GBBz T cells (p=0.0052). IL-21 co-expression significantly enhanced IL-2 production compared to groups lacking IL-21 (21.15.GBBz vs GBBz, p=0.0047; 21.GBBz vs GBBz, p=0.0016). Importantly, GPC3-negative A549 cells did not induce significant effector cytokine production suggesting that the GPC3-CARs in this do not trigger consequential levels of tonic signaling (FIG. 7).

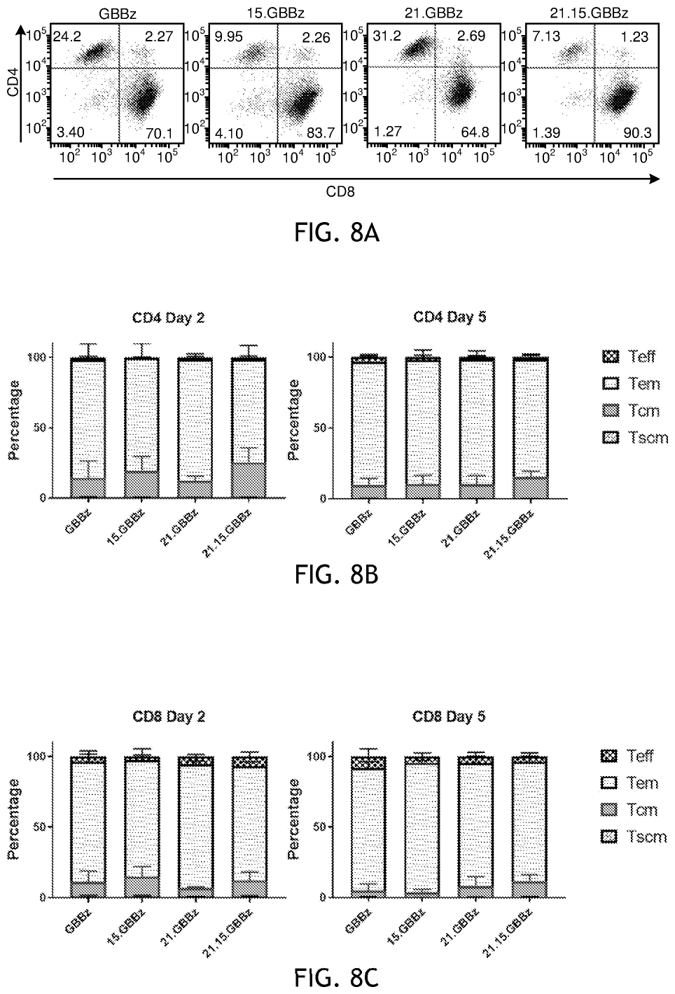

CD4⁺ T cells can produce more effector cytokines than CD8⁺ cells and CD8+ T cell homeostasis is supported by IL-15 which is expressed in 15.GBBz and 21.15.GBBz T cells. To examine if the differences in effector cytokine production were related to differences in CD4/CD8 composition, this parameter was evaluated at baseline and following tumor cell engagement (FIG. 2C). Combined expression of IL-15 and IL-21 increased the CD8⁺ GPC3-CAR T cell population (with a corresponding decrease in the CD4 population) versus GBBz alone at baseline (p=0.0436). Following two rounds of stimulation, the CD8⁺ subset was enriched in 15.GBBz and 21.15.GBBz T cell groups (p=0.0478 and p=0.0078, respectively; FIG. 2C; FIG. 8A).

Overall, GPC3-CAR T cells demonstrate effective GPC3-specific shot-term cytotoxic activity in vitro regardless of cytokine co-expression, the cells undergo IL-15 and/or IL-21-specific changes in both cytokine production profile and CD4/CD8 T cell phenotype distribution that could benefit their in vivo efficacy.

Combined Expression of IL-15 and IL-21 Increases the Proportion of Less Differentiated GPC3-CAR T Cells that Exhibit Enhanced In Vitro Antigen-Dependent Proliferation and Survival.

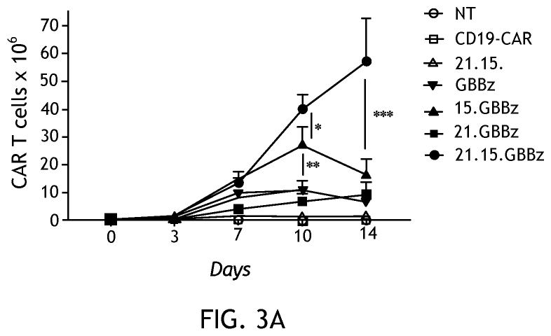

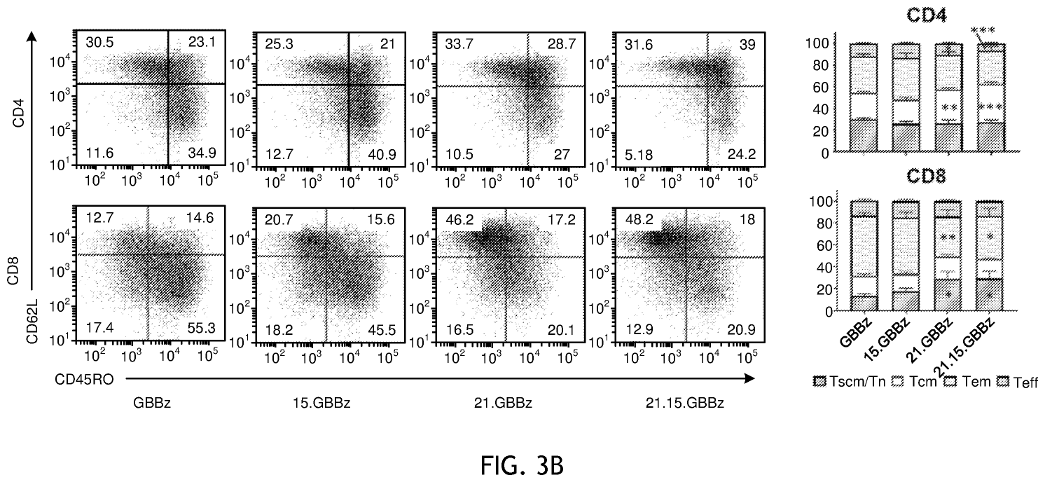

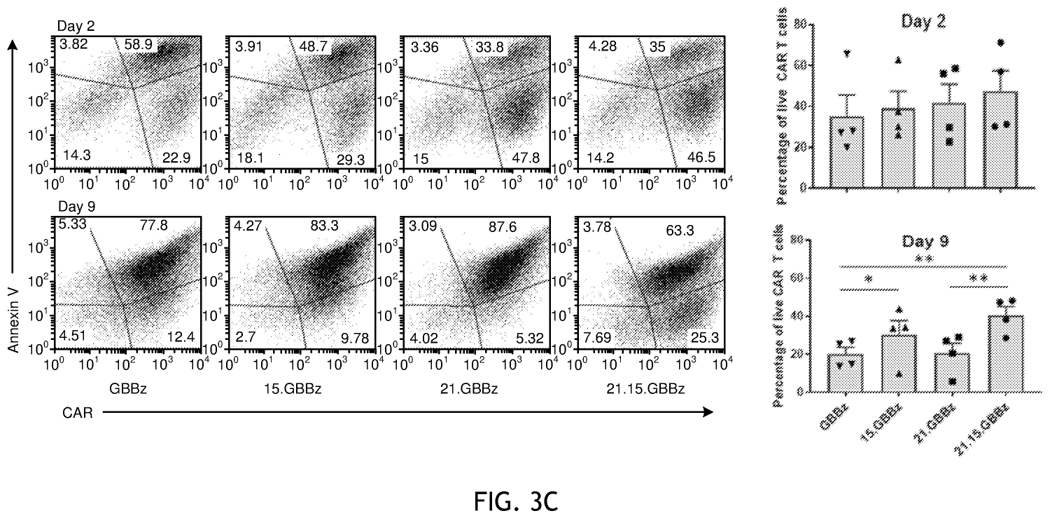

Limited in vivo expansion is a major barrier for effective immunotherapy against solid tumors. To test the proliferative capacity of GPC3-CAR T cells, they were repeatedly exposed to fresh tumor cells in vitro every 3-4 days in the absence of exogenous cytokines. After the second round of stimulation with fresh HCC cells (day 7), T cells expressing GBBz, 15.GBBz, and 21.15.GBBz were beginning to expand to higher numbers than 21.GBBz T cells and control groups (21.15.GBBz/15.GBBz vs 21.GBBz, p<0.001; GBBz vs 21.GBBz, p=0.0486, FIG. 3A). By the end of the third stimulation (day 10), 21.15.GBBz and 15.GBBz T cells expanded significantly more than the GBBz group (15.GBBz vs. GBBz, p=0.0013; 21.15.GBBz vs. 15.GBBz, p=0.0243), suggesting a critical role for IL-15 in enhancing expansion. After the fourth stimulation (day 14), only 21.15.GBBz T cells continued to proliferate, yielding significantly higher cell numbers than all other groups (p<0.001).

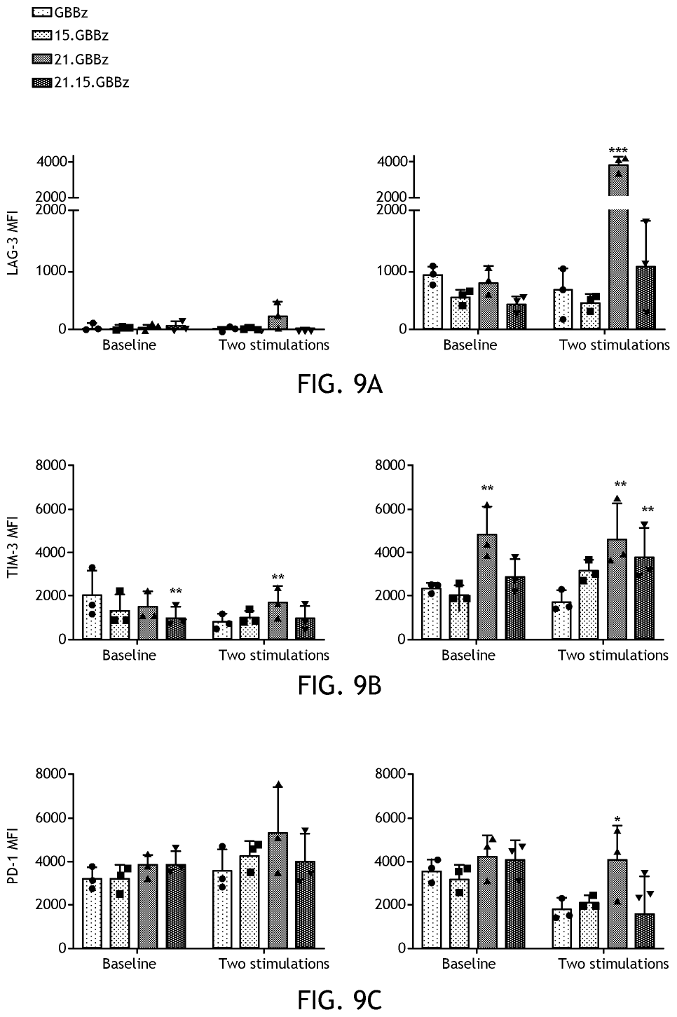

To explore which factors may contribute to the increased proliferative capacity of 21.15.GBBz T cells, the inventor tested for post-manufacturing differences in subset composition among the four CAR T cell groups. Compared to GBBz T cells, both 21.GBBz and 21.15.GBBz T cells displayed a significantly higher proportion of CD4⁺ central memory cells (T_{cm}, CD45RO⁺/CD62L⁺; p=0.0035 and p<0.001, respectively) and of CD8⁺ stem cell memory/naïve cells (T_{scm/Tn}, CD45RO⁻/CD62L⁺; p=0.014 and p=0.012, respectively; FIG. 3B). After stimulation with tumor cells, significant differences in T cell phenotypic subset composition were no longer detected (FIG. 8B and FIG. 8C). Interestingly, although CD8⁺ 21.GBBz T cells had a higher proportion of less differentiated cells, these cells also expressed higher levels of TIM-3 (p=0.0012) at baseline and significantly higher levels of LAG-3, TIM-3, and PD-1 (p<0.001, p=0.001, and p=0.0138, respectively) after stimulation with HCC compared to GBBz cells, indicating an exhausted phenotype (FIG. 9).

Next, it was examined whether the observed differences in proliferation across the GPC3-CAR T cell groups could be related to difference in rates of apoptosis using annexin V staining. Co-expression of IL-15 alone or in combination with IL-21 decreased the rate of apoptosis in T cells resulting in more live cells compared with GBBz alone following three rounds of stimulation with GPC3-positive tumor cells (p=0.0015, as measured on day 9; FIG. 3C). This finding corresponds to the superior expansion of 21.15.GBBz T cells observed in FIG. 3A. Thus, the superior in vitro expansion of GPC3-CAR T cells co-expressing IL-15 and IL-21 is associated with a lower apoptosis rate and increases in Tscm/Tn and Tcm populations.

IL-21 and IL-15 Co-Expression Maintains TCF-1 Expression in GPC3-CAR T Cells.

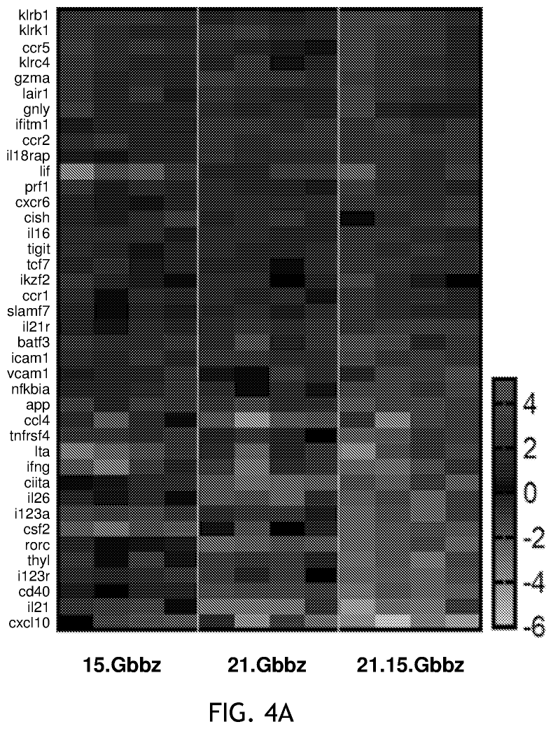

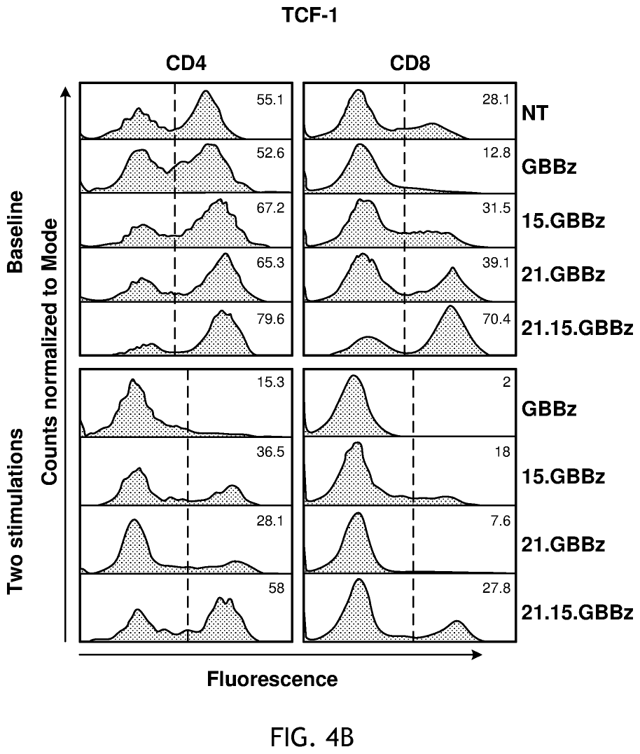

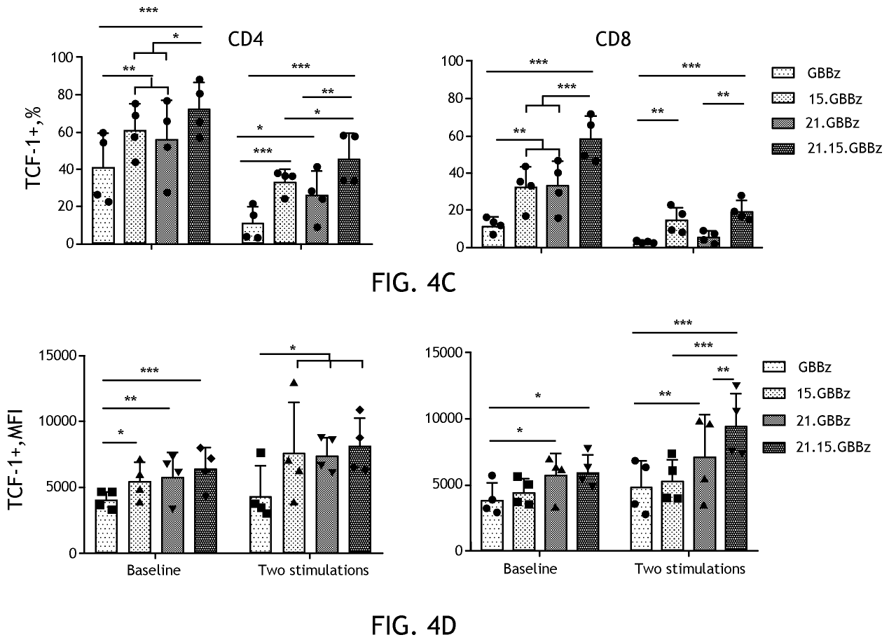

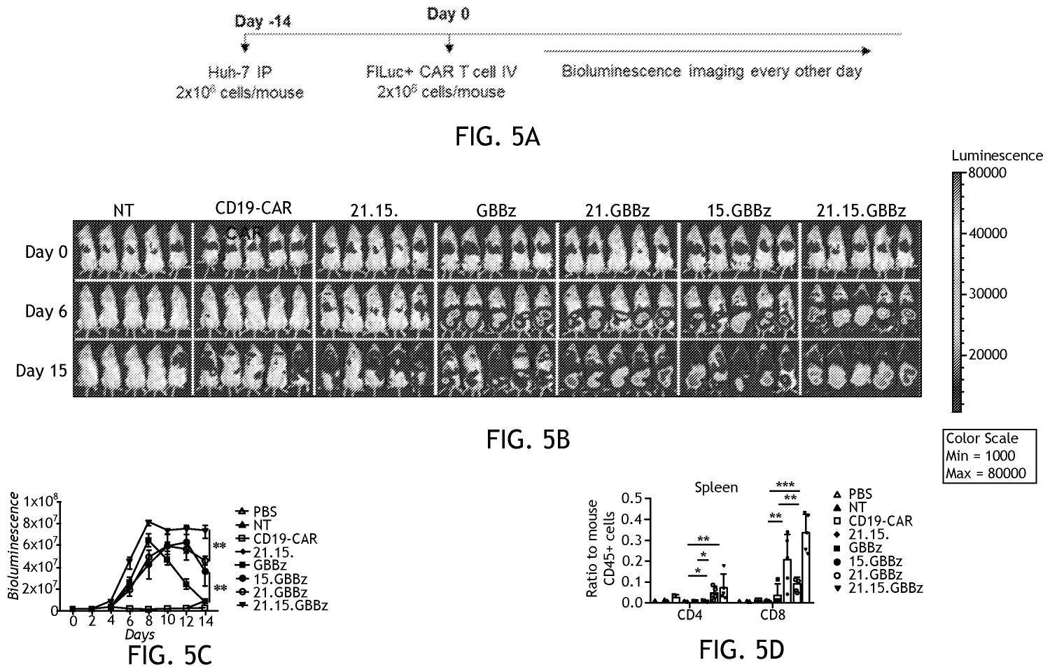

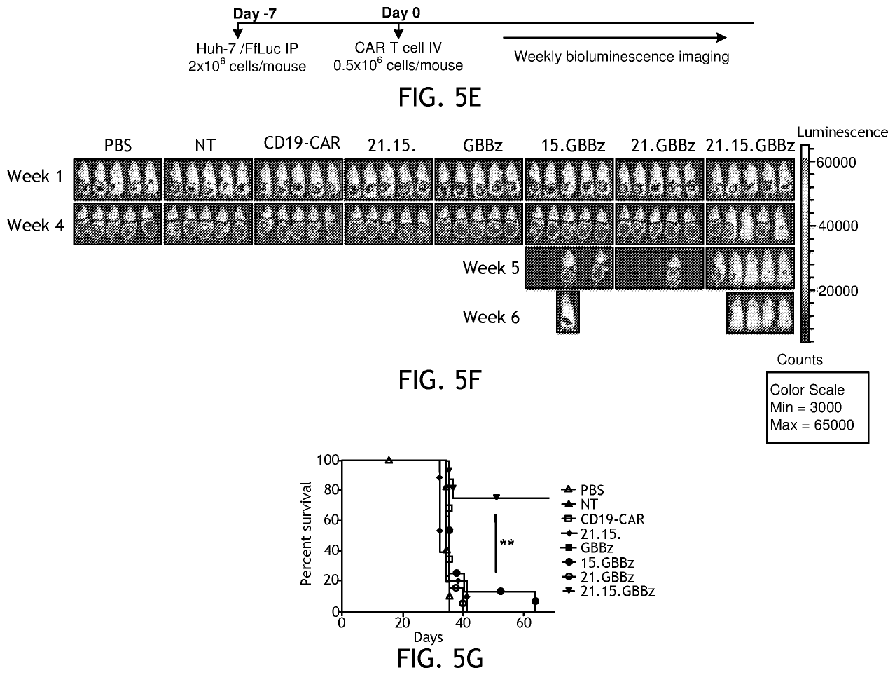

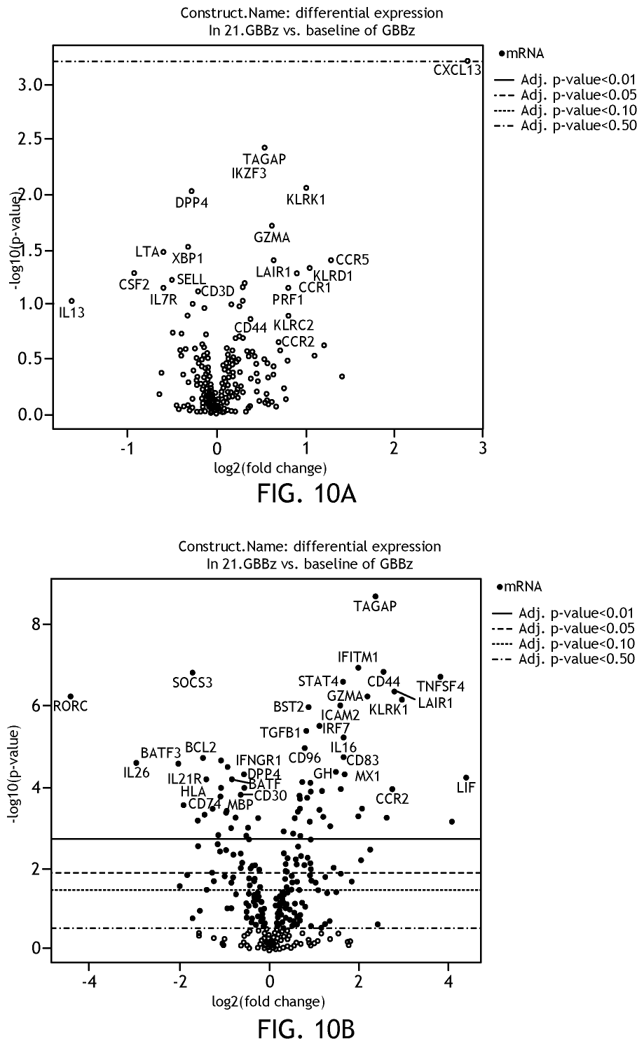

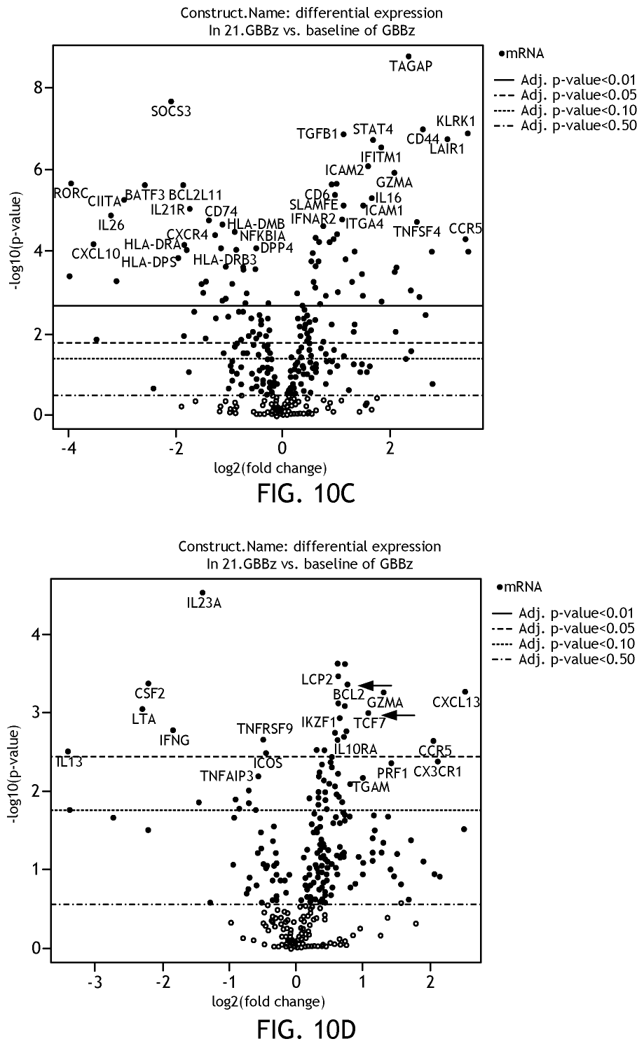

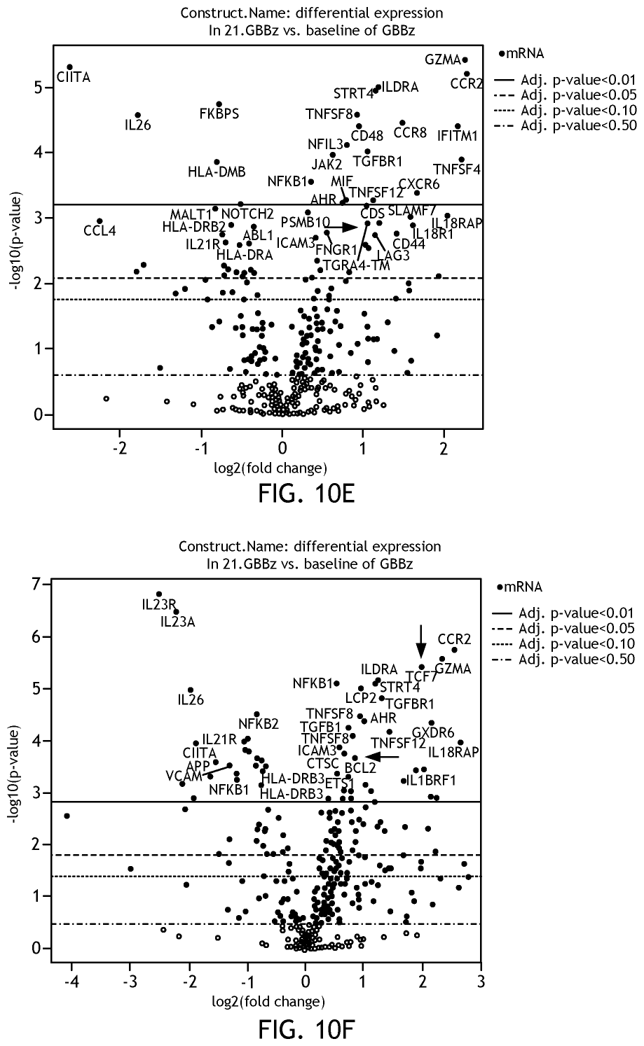

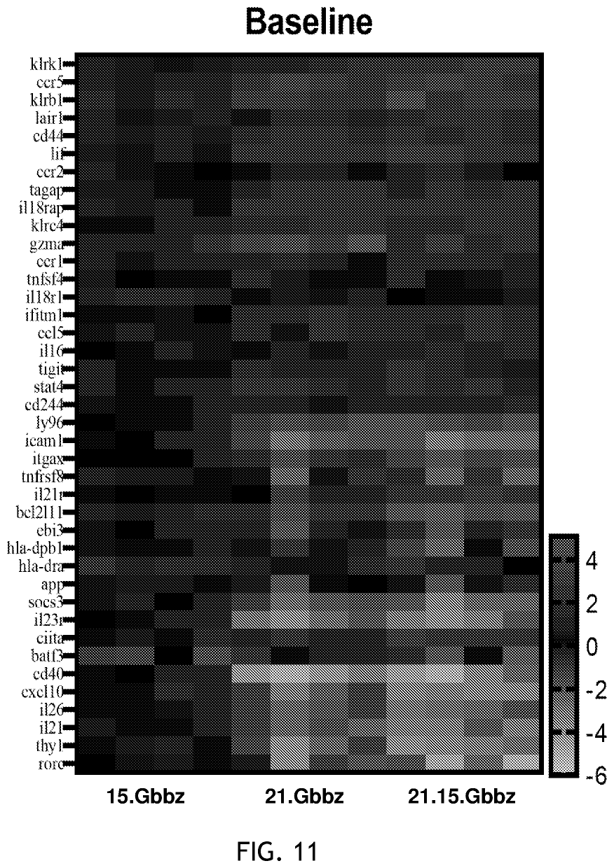

To explore the mechanisms driving differences in proliferation between GPC3-CAR T cell groups, gene expression profiles were examined before and after exposure to GPC3-positive HCC cells. There were significant differences in overall expression patterns at baseline (after manufacturing) in 21.GBBz and 21.15.GBBz T cells compared to GBBz T cells (day 0; FIGS. 10A-10C; FIG. 11). Following stimulation with HCC cells (day 3), the gene expression profiles of 15.GBBz, 21.GBBz and 21.15.GBBz T cells were significantly different than that of GBBz T cells (FIG. 4A; FIGS. 5D-5F;) and global differences included genes related to cytotoxicity (GZMA, GNLY, PRF1), chemotaxis (CCR1, CCR2, CCR5), and apoptosis/survival (BCL-2, TCF7/TCF-1).

The inventor then identified genes related to apoptosis and proliferation with significantly different expression patterns in GPC3-CAR T cells co-expressing cytokines versus GBBz T cells. BCL-2 was overexpressed at the transcriptomic level in 15.GBBz and 21.15.GBBz compared to GBBz T cells (p=0.023 and 0.0025, respectively) (FIGS. 10D-10F), but no difference was detected at the protein level (data not shown) suggesting that BCL-2 does not play a key role in the enhanced survival of 21.15.GBBz T cells. The TCF-1 protein, encoded by TCF7, is a critical transcription factor for T cell development, expansion, and survival (31,32). Compared to GBBz T cells prior to stimulation (day 0), TCF7 was expressed at comparable levels in all GPC3-CAR T cell groups (FIGS. 10A-10C). After stimulation with HCC cells, the gene expression of TCF7 was significantly increased in 21.GBBz (2.2-fold increase, p=0.014), 15.GBBz (2.3-fold increase, p=0.028), and to an even greater extent in 21.15.GBBz T cells (4.1-fold increase, p=0.0003) compared to in GBBz T cells (FIG. 4A). The expression of either IL15, IL-21 alone or in combination improved TCF-1 protein expression in both CD4⁺ and CD8⁺ CAR T cells (FIGS. 4B and 4C). More importantly, the proportion of TCF-1-positive cells at baseline was significantly higher in 21.15.GBBz T cells compared to other CART cell groups (21.15.GBBz vs 15.GBBz in CD4 subset: p=0.0223; in CD8 subset: p<0.001, FIG. 4C). After two consecutive stimulations with tumor cells, the percent of TCF-1-positive cells was the highest in 21.15.GBBz T cell group for the CD4 subset (21.GBBz vs 21.15.GBBz, p=0.002; 15.GBBz vs 21.15.GBBz, p=0.0296) while in the CD8 subset, the 15.GBBz and 21.15.GBBz T cells had the highest percentage of TCF-1-positive cells (15.GBBz vs 21.GBBz p=0.0124; 21.GBBz vs 21.15.GBBz p<0.001; FIG. 4C). The level of TCF-1 expression in the CD8 subset remained highest after two stimulations in the 21.15.GBBz group compared to all other groups (21.GBBz vs 21.15.GBBz, p=0.0044, FIG. 4D).

Co-Expression of IL-15 and IL-21 Enhances In Vivo Expansion, Persistence, and Anti-Tumor Activity of GPC3-CAR T Cells.

To evaluate the in vivo expansion and persistence of GPC3-CAR T cells, HCC xenografts were established in NSG mice and had injected therein T cells co-transduced with the individual GPC3-CAR constructs and an eGFP.Ffluc construct optimized for tracking small numbers of cells in vivo via bioluminescence imaging (FIG. 5A)(30). As observed in a previous study (27), GBBz T cells expanded effectively for eight days, after which the population contracted and disappeared entirely by 15 days post-injection (FIGS. 5B and 5C)(27). 21.GBBz and 15.GBBz T cells had a similar timeline of peak expansion compared to GBBz T cells but persisted longer in vivo before their numbers began to decline (day 12, p<0.001). Co-expression of IL-15 and IL-21 together induced the most robust expansion and persistence of GPC3-CAR T cells in vivo (day 15, 21.15.GBBz vs 21.GBBz p=0.0012). Peripheral blood analysis 15 days after adoptive transfer showed increased frequency of CD8⁺ 15.GBBz and 21.15.GBBz T cells compared to GBBz T cells (p=0.0076 and p<0.001, respectively; FIG. 12). In the spleen, the frequency of CD4⁺ 21.GBBz and 21.15.GBBz CART cells was significantly elevated compared to GBBz T cells (p=0.0386 and p=0.003, respectively). The frequency of CD8⁺ 21.15.GBBz cells was significantly higher in the spleen compared to other groups including 15.GBBz (p=0.0083; FIG. 5D).

As a safety assessment, treated animals were monitored closely for signs of toxicity and serum levels of IL-15 and IL-21 were measured in all therapeutic groups (day 15). No changes in weight or other signs of toxicity were detected that could potentially be associated with cytokines in the serum. IL-21 and -15 serum concentrations in mice treated with cytokine-containing CAR T cells were at levels similar to those of control and GBBz T cell-infused mice at the T cells' peak expansion (FIG. 6B).

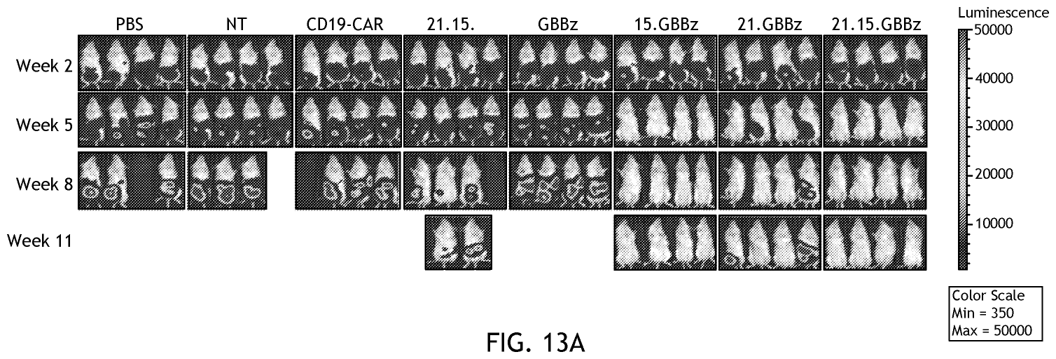

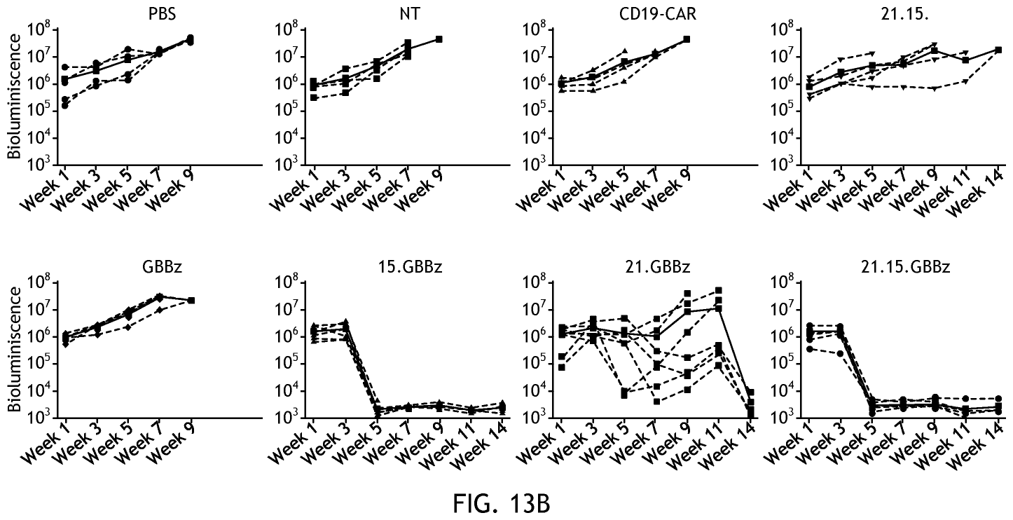

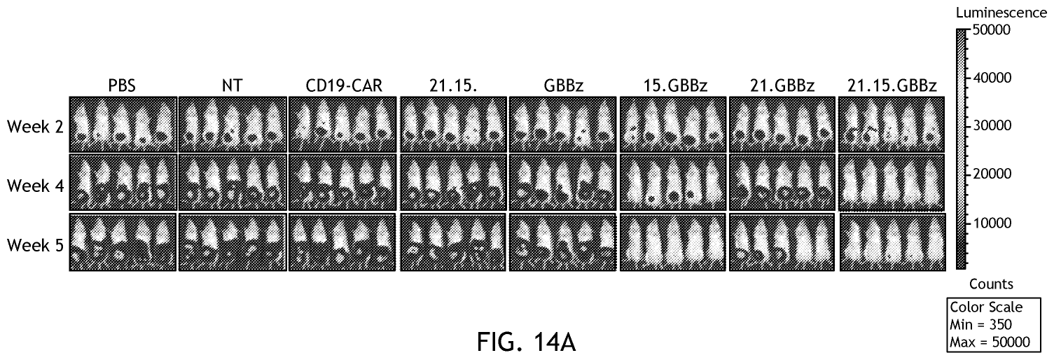

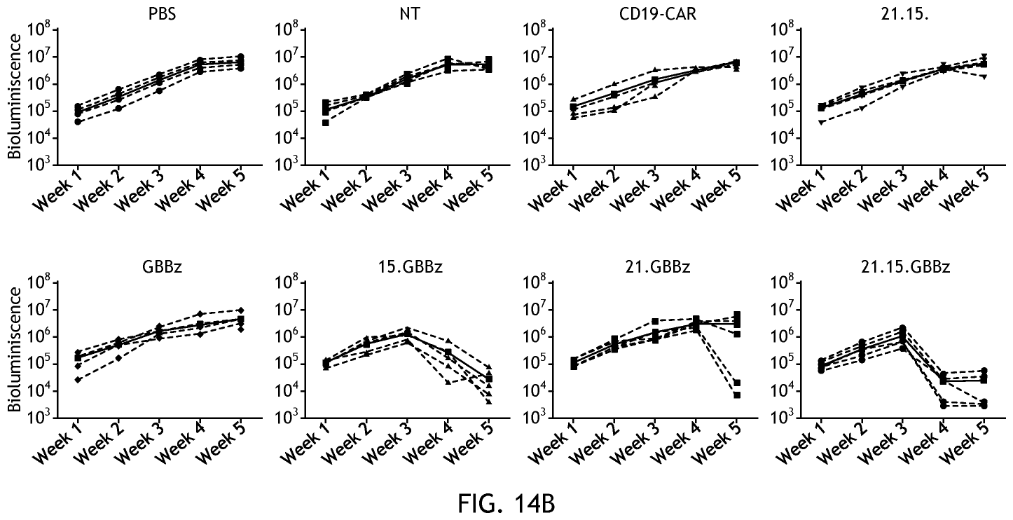

To evaluate the effects of IL-15 and IL-21 co-expression on the antitumor activity of GPC3-CAR T cells, the inventor adoptively transferred these cells into mice bearing Ffluc-labeled tumor xenografts and monitored tumor growth weekly (FIG. 5E). Previously, we established that a single dose of 1×10⁷ GBBz T cells eliminates GPC3⁺ tumor xenografts in NSG mice (27). Therefore, the inventor began with a titrated dose of 5×10⁵ GPC3-CAR T cells per mouse. In a relatively slow-growing GPC3⁺ malignant rhabdoid tumor model, co-expression of IL-15 and/or IL-21 significantly enhanced the antitumor responses after injection of GPC3-CAR T cells (week 5: 21.15.GBBz/15.GBBz vs GBBz, p<0.001; 21.GBBz vs GBBz, p=0.0011, FIG. 13), and IL-15 co-expressing CAR T cell groups induced a more rapid anti-tumor effect (week 5: 21.15.GBBz/15.GBBz vs 21.GBBz, p<0.001). Next, GPC3-CAR T cell antitumor responses were examined in a rapidly growing HCC xenograft model injecting 2×10⁶ CAR T cells. It was determined that 15.GBBz and 21.15.GBBz T cells mediated superior antitumor activity compared to GBBz or 21.GBBz T cells and control groups (p<0.001; FIG. 14). In this model, 21.15.GBBz T cells eliminated tumors more rapidly than 15.GBBz T cells (week 4, p<0.001). To determine how additional stress would affect antitumor activity of GPC3-CAR T cells, the inventor injected a low dose of 5×10⁵ CAR T cells in mice engrafted with rapidly growing HCC xenografts. At this dose, only 21.15.GBBz T cells, but not 15.GBBz T cells, maintained antitumor activity, which translated into significant survival advantage (15.GBBz vs 21.15.GBBz, p=0.0014, FIGS. 5F and 5G). These results demonstrate that 21.15.GBBz T cells have superior expansion, persistence and antitumor activity against HCC in vivo.

### Significance of Certain Embodiments

Shown herein is evidence that GPC3-CAR T cells co-expressing IL15 and IL-21 have superior expansion and antitumor activity in preclinical models of HCC compared to CAR T cells with either cytokine alone or without cytokine co-expression. Insight is provided into the broad gene expression changes related to transgenic expression of IL-15 and IL-21 and TCF-1 is identified as a key transcription factor associated with the enhanced proliferative capacity of 21.15.GBBz T cells.

Co-expression of IL-21 and/or IL-15 with GBBz did not impact the potent, specific, short-term in vitro cytolytic activity of T cells against HCC tumors cells. These results indicate that IL15 and/or IL-21 co-expression in GPC3-CAR T cells does not interfere with the transmission of an activation signal from the CD3 domain to the T cells' cytolytic machinery. In contrast, there were significant differences in effector cytokine production polarization when comparing GBBz T cells to those co-expressing one or both cytokines. As in a previous study, GBBz T cells secreted a T_{H}1-polarized cytokine profile (high IFN-γ and GM-CSF; low IL-10 and IL-4)(27). This overall trend was recapitulated in the present disclosure, with CAR T cell groups showing GBBz-mediated T_{H}1 polarization regardless of cytokine co-expression. However, there was a striking decrease in IL-13 production in CAR T cells co-expressing IL-15 (15.GBBz and 21.15.GBBz T cells). IL-13 is a T_{H}2-cytokine primarily produced by CD4⁺ T cells that generates many of the same biological effects as IL-4, including decreasing the antitumor function of T cells and promoting tumor cell proliferation (33,34). IL-13 also plays an important role in homeostasis of myeloid-derived suppressor cells (MDSCs), which can dampen the efficacy of immunotherapies, increase metastasis formation, cancer progression and inhibit CAR T cell activity(34-38); therefore, limiting the amount of IL-13 in the tumor microenvironment may enhance the therapeutic potential of CAR T cells (38,39). Thus, GPC3-CAR T cells co-expressing IL-15 alone or in combination with IL-21 in specific embodiments provides a further therapeutic advantage by decreasing tumor cell proliferation and ameliorating direct and MDSC-mediated immunosuppressive effects, leading to better antitumor activity in the clinical setting.

A key objective of this disclosure was to enhance the expansion and persistence of GPC3-CAR T cells following tumor cell engagement. Co-expression of IL-15 and IL-21 in GPC3-CAR T cells achieves this goal in both in vitro and in vivo through at least three distinct mechanisms. First, co-expression of IL-21 increases the proportion of naïve/stem cell memory and central memory GPC3-CAR T cells post-manufacture. These less-differentiated T cells have greater proliferative capacity than more mature cells (40-42), providing a potential proliferative advantage for GPC3-CAR T cells co-expressing IL-21. Given that all experimental groups were manufactured under the same culture conditions including supplementation with IL-15 and IL-21, this finding was unexpected. Continuous production of IL-21 via transgenic expression from the GPC3-CAR throughout the culturing process likely influenced the T cell phenotype. Second, compared to other experimental groups co-expression of IL-15 alone or in combination with IL-21 decreased the proportion of transduced T cells undergoing apoptosis after multiple in vitro stimulations with HCC tumor cells, thus proportionately increasing surviving CAR T cells. Finally, the tcf-7 gene encoding TCF-1—a key transcription factor in T cell development, expansion, memory formation, and survival (31,32,43,44)—was expressed at a higher level in the CD8⁺ subsets of GPC3-CAR T cells expressing IL-15, IL-21, or both compared to in CD8⁺ GBBz T cells. However, following two rounds of stimulation with HCC cells, TCF-1 expression was maintained at the highest level in 21.15.GBBz T cells and was associated with enrichment for and continued expansion of CD8⁺ CAR T cells. The findings indicate that expression of IL-21 alone or in combination with IL-15 during initial manufacturing can enrich for less differentiated T cells with a greater propensity for proliferation, but IL-15 is required to protect against apoptosis. Additionally, TCF-1 appears to play a role in enhancing the expansion and survival of IL-15- and IL-21-co-expressing GPC3-CAR T cells.

In cancer patients, the effector to target ratio of CAR T cells to cancer cells greatly favors cancer; thus, its complete elimination in patients will likely require CAR T cells to kill repeatedly and expand. In sequential killing assays, in which GPC3-CAR T cells are repeatedly exposed to fresh tumor cells in vitro, combined expression of IL-15 and -21 resulted in the most expansion. Additionally, in an aggressive xenograft model of HCC treated with the lowest dose of GPC3-CAR T cells thereby stressing their expansion capacity, cells transduced with 21.15.GBBz resulted in significant survival advantage. These findings strongly indicate that IL-15 and IL-21 should provide a potent antitumor activity in the clinical setting.

Safety remains a central requirement for all cancer treatments. Elevated levels of IL-15 and IL-21 can cause side effects as described in early phase studies of subcutaneous or intravenous recombinant IL-21 and IL-15 administration in patients with cancer (45). In these studies, at the maximum tolerated dose (MTD), peripheral blood peak concentrations of IL-15 and IL-21 were 1608 pg/ml and 141 ng/ml, respectively (46,47). In mice infused with GPC3-CAR T cells expressing IL-15 and/or IL-21, cytokine concentrations at the peak of T cell expansion remained 100-1000-fold below the peak levels at corresponding MTDs measured in humans. Therefore, in particular embodiments there are no systemic toxicities in patients treated with GPC3-CAR T cells co-expressing IL-15 and/or IL-21. Nevertheless, use of a suicide gene-based system such as inducible caspase 9 to eliminate therapeutic cells if necessary may be utilized (48).

In conclusion, there is shown an effective approach to treat HCC with GPC3-CAR T cells co-expressing IL-15 and IL-21. The findings address a major barrier by enhancing the expansion and persistence of therapeutic cells, resulting robust antitumor responses.

### REFERENCES

All patents and publications mentioned in the specification are indicative of the level of those skilled in the art to which the invention pertains. All patents and publications are herein incorporated by reference in their entirety to the same extent as if each individual publication was specifically and individually indicated to be incorporated by reference.
1. Villanueva A. Hepatocellular Carcinoma. N Engl J Med 2019; 380(15):1450-62 doi 10.1056/NEJMra1713263.
2. Maude S L, Frey N, Shaw P A, Aplenc R, Barrett D M, Bunin N J, et al. Chimeric antigen receptor T cells for sustained remissions in leukemia. N Engl J Med 2014; 371(16):1507-17 doi 10.1056/NEJMoa1407222.
3. Davila M L, Riviere I, Wang X, Bartido S, Park J, Curran K, et al. Efficacy and toxicity management of 19-28z CAR T cell therapy in B cell acute lymphoblastic leukemia. Sci Transl Med 2014; 6(224):224ra25 doi 10.1126/scitranslmed.3008226.
4. Turtle C J, Hanafi L A, Berger C, Gooley T A, Cherian S, Hudecek M, et al. CD19 CAR-T cells of defined CD4+:CD8+ composition in adult B cell ALL patients. J Clin Invest 2016; 126(6):2123-38 doi 10.1172/JCI85309.
5. Turtle C J, Hanafi L A, Berger C, Hudecek M, Pender B, Robinson E, et al. Immunotherapy of non-Hodgkin's lymphoma with a defined ratio of CD8+ and CD4+CD19-specific chimeric antigen receptor-modified T cells. Sci Transl Med 2016; 8(355):355ra116 doi 10.1126/scitranslmed.aaf8621.
6. Lee D W, Kochenderfer J N, Stetler-Stevenson M, Cui Y K, Delbrook C, Feldman S A, et al. T cells expressing CD19 chimeric antigen receptors for acute lymphoblastic leukaemia in children and young adults: a phase 1 dose-escalation trial. Lancet 2015; 385(9967):517-28 doi 10.1016/S0140-6736(14)61403-3.
7. Bo Zhai D S, Huiping Gao, Xingxing Qi, Hua Jiang, Yuan Zhang, Jiachang Chi, Huaying Ruan, Huamao Wang, Qinhua Cindy Ru, Zonghai Li. A phase I study of anti-GPC3 chimeric antigen receptor modified T cells (GPC3 CAR-T) in Chinese patients with refractory or relapsed GPC3+ hepatocellular carcinoma (r/r GPC3+ HCC). J Clin Oncol 2017; 35(2017 (suppl; abstr 3049)).
8. Heczey A, Louis C U, Savoldo B, Dakhova O, Durett A, Grilley B, et al. CAR T Cells Administered in Combination with Lymphodepletion and PD-1 Inhibition to Patients with Neuroblastoma. Molecular therapy: the journal of the American Society of Gene Therapy 2017; 25(9):2214-24 doi 10.1016/j.ymthe.2017.05.012.
9. Ahmed N, Brawley V, Hegde M, Bielamowicz K, Kalra M, Landi D, et al. HER2-Specific Chimeric Antigen Receptor-Modified Virus-Specific T Cells for Progressive Glioblastoma: A Phase 1 Dose-Escalation Trial. JAMA Oncol 2017 doi 10.1001/jamaoncol.2017.0184.
10. Ahmed N, Brawley V S, Hegde M, Robertson C, Ghazi A, Gerken C, et al. Human Epidermal Growth Factor Receptor 2 (HER2)-Specific Chimeric Antigen Receptor-Modified T Cells for the Immunotherapy of HER2-Positive Sarcoma. J Clin Oncol 2015; 33(15):1688-96 doi 10.1200/JCO.2014.58.0225.
11. Park J R, Digiusto D L, Slovak M, Wright C, Naranjo A, Wagner J, et al. Adoptive transfer of chimeric antigen receptor re-directed cytolytic T lymphocyte clones in patients with neuroblastoma. Molecular therapy: the journal of the American Society of Gene Therapy 2007; 15(4):825-33 doi 10.1038/sj.mt.6300104.
12. Louis C U, Savoldo B, Dotti G, Pule M, Yvon E, Myers G D, et al. Antitumor activity and long-term fate of chimeric antigen receptor-positive T cells in patients with neuroblastoma. Blood 2011; 118(23):6050-6 doi 10.1182/blood-2011-05-354449.
13. Pilipow K, Roberto A, Roederer M, Waldmann T A, Mavilio D, Lugli E. IL15 and T-cell Stemness in T-cell-Based Cancer Immunotherapy. Cancer Res 2015; 75(24):5187-93 doi 10.1158/0008-5472.Can-15-1498.
14. Davis M R, Zhu Z, Hansen D M, Bai Q, Fang Y. The role of IL-21 in immunity and cancer. Cancer Lett 2015; 358(2):107-14 doi 10.1016/j.canlet.2014.12.047.
15. Hoyos V, Savoldo B, Quintarelli C, Mahendravada A, Zhang M, Vera J, et al. Engineering CD19-specific T lymphocytes with interleukin-15 and a suicide gene to enhance their anti-lymphoma/leukemia effects and safety. Leukemia 2010; 24(6):1160.
16. Markley J C, Sadelain M. IL-7 and IL-21 are superior to IL-2 and IL-15 in promoting human T cell-mediated rejection of systemic lymphoma in immunodeficient mice. Blood 2010; 115(17):3508-19.
17. Krenciute G, Prinzing B L, Yi Z, Wu M F, Liu H, Dotti G, et al. Transgenic Expression of IL15 Improves Antiglioma Activity of IL13Ralpha2-CAR T Cells but Results in Antigen Loss Variants. Cancer Immunol Res 2017; 5(7):571-81 doi 10.1158/2326-6066.CIR-16-0376.
18. Chen Y, Sun C, Landoni E, Metelitsa L S, Dotti G, Savoldo B. Eradication of neuroblastoma by T cells redirected with an optimized GD2-specific chimeric antigen receptor and interleukin-15. Clinical cancer research: an official journal of the American Association for Cancer Research 2019 doi 10.1158/1078-0432.Ccr-18-1811.
19. Strengell M, Matikainen S, Siren J, Lehtonen A, Foster D, Julkunen I, et al. IL-21 in synergy with IL-15 or IL-18 enhances IFN-gamma production in human N K and T cells. J Immunol 2003; 170(11):5464-9.
20. Zeng R, Spolski R, Finkelstein S E, Oh S, Kovanen P E, Hinrichs C S, et al. Synergy of IL-21 and IL-15 in regulating CD8+ T cell expansion and function. J Exp Med 2005; 201(1):139-48 doi 10.1084/jem.20041057.
21. Chan E S, Pawel B R, Corao D A, Venneti S, Russo P, Santi M, et al. Immunohistochemical expression of glypican-3 in pediatric tumors: an analysis of 414 cases. Pediatr Dev Pathol 2013; 16(4):272-7 doi 10.2350/12-06-1216-(M A.
22. Toretsky J A, Zitomersky N L, Eskenazi A E, Voigt R W, Strauch E D, Sun C C, et al. Glypican-3 expression in Wilms tumor and hepatoblastoma. J Pediatr Hematol Oncol 2001; 23(8):496-9.
23. Yamauchi N, Watanabe A, Hishinuma M, Ohashi Ki, Midorikawa Y, Morishita Y, et al. The glypican 3 oncofetal protein is a promising diagnostic marker for hepatocellular carcinoma. Mod Pathol 2005; 18(12):1591-8.
24. Haruyama Y, Kataoka H. Glypican-3 is a prognostic factor and an immunotherapeutic target in hepatocellular carcinoma. World J Gastroenterol 2016; 22(1):275-83 doi 10.3748/wjg.v22i1.275.
25. Kinoshita Y, Tanaka S, Souzaki R, Miyoshi K, Kohashi K, Oda Y, et al. Glypican 3 expression in pediatric malignant solid tumors. Eur J Pediatr Surg 2015; 25(1):138-44 doi 10.1055/s-0034-1393961.
26. Gao H, Li K, Tu H, Pan X, Jiang H, Shi B, et al. Development of T cells redirected to glypican-3 for the treatment of hepatocellular carcinoma. Clinical cancer research: an official journal of the American Association for Cancer Research 2014; 20(24):6418-28 doi 10.1158/1078-0432.CCR-14-1170.
27. Li W, Guo L, Rathi P, Marinova E, Gao X, Wu M F, et al. Redirecting T Cells to Glypican-3 with 4-1BB Zeta Chimeric Antigen Receptors Results in Th1 Polarization and Potent Antitumor Activity. Human gene therapy 2017; 28(5):437-48 doi 10.1089/hum.2016.025.
28. Iwahori K, Kakarla S, Velasquez M P, Yu F, Yi Z, Gerken C, et al. Engager T cells: a new class of antigen-specific T cells that redirect bystander T cells. Molecular therapy: the journal of the American Society of Gene Therapy 2015; 23(1):171-8 doi 10.1038/mt.2014.156.
29. Ramos C A, Rouce R, Robertson C S, Reyna A, Narala N, Vyas G, et al. In Vivo Fate and Activity of Second- versus Third-Generation CD19-Specific CAR-T Cells in B Cell Non-Hodgkin's Lymphomas. Molecular therapy: the journal of the American Society of Gene Therapy 2018; 26(12):2727-37 doi 10.1016/j.ymthe.2018.09.009.
30. Rabinovich B A, Ye Y, Etto T, Chen J Q, Levitsky H I, Overwijk W W, et al. Visualizing fewer than 10 mouse T cells with an enhanced firefly luciferase in immunocompetent mouse models of cancer. Proc Natl Acad Sci USA 2008; 105(38):14342-6 doi 10.1073/pnas.0804105105.
31. Weber B N, Chi A W, Chavez A, Yashiro-Ohtani Y, Yang Q, Shestova O, et al. A critical role for TCF-1 in T-lineage specification and differentiation. Nature 2011; 476(7358):63-8 doi 10.1038/nature10279.
32. Kratchmarov R, Magun A M, Reiner S L. TCF1 expression marks self-renewing human CD8(+) T cells. Blood advances 2018; 2(14):1685-90 doi 10.1182/bloodadvances.2018016279.
33. Park J M, Terabe M, Donaldson D D, Forni G, Berzofsky J A. Natural immunosurveillance against spontaneous, autochthonous breast cancers revealed and enhanced by blockade of IL-13-mediated negative regulation. Cancer immunology, immunotherapy: CII 2008; 57(6):907-12 doi 10.1007/s00262-007-0414-0.
34. Terabe M, Park J M, Berzofsky J A. Role of IL-13 in regulation of anti-tumor immunity and tumor growth. Cancer immunology, immunotherapy: CII 2004; 53(2):79-85 doi 10.1007/s00262-003-0445-0.
35. Kapanadze T, Gamrekelashvili J, Ma C, Chan C, Zhao F, Hewitt S, et al. Regulation of accumulation and function of myeloid derived suppressor cells in different murine models of hepatocellular carcinoma. Journal of hepatology 2013; 59(5):1007-13 doi 10.1016/j.jhep.2013.06.010.
36. Yu S J, Ma C, Heinrich B, Brown Z J, Sandhu M, Zhang Q, et al. Targeting the crosstalk between cytokine-induced killer cells and myeloid-derived suppressor cells in hepatocellular carcinoma. Journal of hepatology 2019; 70(3):449-57 doi 10.1016/j.jhep.2018.10.040.
37. Sinha P, Clements V K, Ostrand-Rosenberg S. Interleukin-13-regulated M2 Macrophages in Combination with Myeloid Suppressor Cells Block Immune Surveillance against Metastasis. Cancer Research 2005; 65(24):11743-51 doi 10.1158/0008-5472.Can-05-0045.
38. Long A H, Highfill S L, Cui Y, Smith J P, Walker A J, Ramakrishna S, et al. Reduction of MDSCs with All-trans Retinoic Acid Improves CAR Therapy Efficacy for Sarcomas. Cancer Immunol Res 2016; 4(10):869-80 doi 10.1158/2326-6066.Cir-15-0230.
39. Parihar R, Rivas C, Huynh M, Omer B, Lapteva N, Metelitsa L S, et al. N K Cells Expressing a Chimeric Activating Receptor Eliminate MDSCs and Rescue Impaired CAR-T Cell Activity against Solid Tumors. Cancer Immunol Res 2019; 7(3):363-75 doi 10.1158/2326-6066.Cir-18-0572.
40. Terakura S, Yamamoto T N, Gardner R A, Turtle C J, Jensen M C, Riddell S R. Generation of CD19-chimeric antigen receptor modified CD8+ T cells derived from virus-specific central memory T cells. Blood 2012; 119(1):72-82 doi 10.1182/blood-2011-07-366419.
41. Klebanoff C A, Gattinoni L, Torabi-Parizi P, Kerstann K, Cardones A R, Finkelstein S E, et al. Central memory self/tumor-reactive CD8+ T cells confer superior antitumor immunity compared with effector memory T cells. Proc Natl Acad Sci USA 2005; 102(27):9571-6 doi 10.1073/pnas.0503726102.
42. Gattinoni L, Lugli E, Ji Y, Pos Z, Paulos C M, Quigley M F, et al. A human memory T cell subset with stem cell-like properties. Nature medicine 2011; 17(10):1290-7 doi 10.1038/nm.2446.
43. Gautam S, Fioravanti J, Zhu W, Le Gall J B, Brohawn P, Lacey N E, et al. The transcription factor c-Myb regulates CD8(+) T cell stemness and antitumor immunity. Nat Immunol 2019; 20(3):337-49 doi 10.1038/s41590-018-0311-z.
44. Xing S, Li F, Zeng Z, Zhao Y, Yu S, Shan Q, et al. Tcf1 and Lef1 transcription factors establish CD8(+) T cell identity through intrinsic HDAC activity. Nat Immunol 2016; 17(6):695-703 doi 10.1038/ni.3456.
45. Spolski R, Leonard W J. Interleukin-21: a double-edged sword with therapeutic potential. Nat Rev Drug Discov 2014; 13(5):379-95 doi 10.1038/nrd4296.
46. Thompson J A, Curti B D, Redman B G, Bhatia S, Weber J S, Agarwala S S, et al. Phase I study of recombinant interleukin-21 in patients with metastatic melanoma and renal cell carcinoma. J Clin Oncol 2008; 26(12):2034-9 doi 10.1200/JCO.2007.14.5193.
47. Conlon K C, Lugli E, Welles H C, Rosenberg S A, Fojo A T, Morris J C, et al. Redistribution, hyperproliferation, activation of natural killer cells and CD8 T cells, and cytokine production during first-in-human clinical trial of recombinant human interleukin-15 in patients with cancer. J Clin Oncol 2015; 33(1):74-82 doi 10.1200/jco.2014.57.3329.
48. Di Stasi A, Tey S K, Dotti G, Fujita Y, Kennedy-Nasser A, Martinez C, et al. Inducible apoptosis as a safety switch for adoptive cell therapy. N Engl J Med 2011; 365(18):1673-83 doi 10.1056/NEJMoa1106152.

Although the present disclosure and its advantages have been described in detail, it should be understood that various changes, substitutions and alterations can be made herein without departing from the spirit and scope of the design as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the present disclosure, processes, machines, manufacture, compositions of matter, means, methods, or steps, presently existing or later to be developed that perform substantially the same function or achieve substantially the same result as the corresponding embodiments described herein may be utilized according to the present disclosure. Accordingly, the appended claims are intended to include within their scope such processes, machines, manufacture, compositions of matter, means, methods, or steps.

What is claimed is:

1. An isolated T cell, wherein said T cell comprises a chimeric antigen receptor that targets a tumor antigen, comprises a recombinant T cell receptor that targets a tumor antigen, is viral-specific, and/or is tumor antigen-specific, wherein said cell comprises
one or both of:
(i) at least one recombinant interleukin (IL), and
(ii) induced expression of at least one endogenous IL,
wherein the interleukin is two or more of IL-7, IL-2, IL-12, IL-15, IL-21, and IL-18.

2. The cell of claim 1, wherein the interleukin is at least IL-15.

3. The cell of claim 1, wherein the interleukin is at least IL-21.

4. The cell of any one of claims 1-3, wherein the interleukin is the combination of IL-15 and IL-21.

5. The cell of any one of claims 1-4, wherein the chimeric antigen receptor is expressed from a recombinant nucleic acid.

6. The cell of claim 5, wherein the recombinant nucleic acid is a vector.

7. The cell of claim 6, wherein the vector is a viral vector or non-viral vector.

8. The cell of claim 7, wherein the viral vector is an adenoviral vector, lentiviral vector, retroviral vector, or adeno-associated viral vector.

9. The cell of any one of claims 1-8, wherein IL-15, IL-21, or a combination thereof are expressed from a recombinant nucleic acid and/or from an endogenous gene that is under the transcriptional control of a recombinantly modified promoter region.

10. The cell of claim 9, wherein the recombinant IL-15, IL-21, or combination thereof are expressed from a recombinant nucleic acid.

11. The cell of claim 10, wherein the recombinant nucleic acid is a vector.

12. The cell of claim 11, wherein the vector is a viral vector or a non-viral vector.

13. The cell of any one of claims 1-12, wherein the tumor antigen-specific CAR is expressed from a recombinant nucleic acid.

14. The cell of claim 13, wherein the recombinant nucleic acid is a vector.

15. The cell of claim 13 or 14, wherein the recombinant nucleic acid from which the tumor antigen-specific CAR is expressed is the same molecule as the recombinant nucleic acid from which one or more interleukins are expressed.

16. The cell of claim 15, wherein the nucleic acid comprises a cleavable linker between the tumor antigen-specific CAR and the one or more interleukins to produce independent expression of CAR and the one or more interleukins.

17. The cell of any one of claims 1-16, wherein the tumor antigen-specific CAR comprises one, two, three, or costimulatory domains.

18. The cell of claim 17, wherein the costimulatory domain is selected from the group consisting of CD28, 4-1BB, OX40, DAP10, DAP12, CD27, ICOS, and a combination thereof.

19. The cell of any one of claims 1-18, wherein the induced expression of at least one endogenous IL in the cell comprises recombinant genome editing of at least one regulatory region of the endogenous IL.

20. The cell of claim 19, wherein the recombinant genome editing comprises Zinc finger nucleases (ZFNs), Transcription Activator-Like Effector Nucleases (TALENs), the CRISPR/Cas9 system, and/or engineered meganuclease re-engineered homing endonucleases.

21. The cell of any one of claims 1-20, wherein the T cells are virus-specific T cells.

22. The cell of claim 21, wherein the virus is BK Virus, Human Herpesvirus 6, Cytomegalovirus, Epstein-Barr Virus, Hepatitis B virus, Hepatitis C virus, or Adenovirus.

23. The cell of any one of claims 1-22, wherein the chimeric antigen receptor targets glypican-3 (GPC3).

24. The cell of any one of claims 1-23, wherein the cell is a T cell, the chimeric antigen receptor targets GPC3, and the cytokine is both IL-15 and IL-21.

25. An isolated population of cells, comprising a plurality of any one of the cells of any one of claims 1-24.

26. The population of claim 25, wherein the majority of cells in the population are the cells of any one of claims 1-24.

27. The population of any one of claims 25-26, wherein greater than 70, 75, 80, 85, 90, 91, 92, 93, 94, 95, 96, 97, 98, or 99% of cells in the population are the cells of any one of claims 1-24.

28. A composition comprising the population of cells of any one of claim 25, 26, or 27.

29. The composition of claim 28, wherein the composition is in a pharmaceutically acceptable excipient.

30. The composition of claim 28 or 29, wherein the population of cells is in a solution that is sterile, non-pyogenic, and isotonic.

31. The composition of any one of claims 28-30, wherein the composition is frozen.

32. A method of inhibiting proliferation and/or activity of tumor antigen-positive cells in an individual, comprising the step of providing to the individual a therapeutically effective amount of a plurality of the cells of any one of claim 1-24.

33. The method of claim 32, wherein the tumor antigen is GPC3.

34. The method of claim 32 or 33, wherein the cancer cells are hepatocellular carcinoma cells, liver cancer cells, embryonal sarcoma cells, rhabdoid tumor cells, Wilms tumor cells, choriocarcinoma cells, or yolk sac tumor cells.

35. The method of any one of claims 32-34, wherein the individual is receiving, has received and/or will receive one or more additional cancer therapies.

36. The method of any one of claims 32-35, wherein the cells are provided systemically or locally.

37. The method of claim 36, wherein the cells are systemically or locally provided by injection.

38. The method of any one of claims 32-37, wherein the cells are provided to the individual more than once.

39. The method of any one of claims 32-38, wherein the individual has hepatoblastoma, hepatocellular carcinoma, malignant rhabdoid tumors, yok sac tumors, undifferentiated sarcoma of the liver, liposarcoma, Wilm's tumor, or choriocarcinoma.

40. A method of enhancing a T cell therapy, comprising the step of modifying the T cells to express:
(a) recombinant IL-15 and IL-21,
(b) induced expression of endogenous IL-15 and IL-21,
(c) both (a) and (b), or
(d) recombinant IL-15 or IL-21, and induced expression of endogenous IL-21 or IL-15, respectively,
wherein after modifying at least some of the T cells are protected from apoptosis following exposure to cancer cells and/or wherein the T cells have enhanced in vivo expansion and persistence compared to T cells without the modifying.

41. The method of claim 40, wherein the T cell therapy comprises T cells modified to express one or more engineered antigen receptors.

42. The method of claim 41, wherein the engineered antigen receptor comprises a chimeric antigen receptor, a T cell receptor, or both.

43. The method of any one of claims 40-42, wherein the T cell therapy comprises T cells modified to express a chimeric antigen receptor that targets GPC3.

44. A method of protecting T cells of a T cell therapy from apoptosis upon exposure to cancer cells, comprising the step of modifying the T cells to express:
(a) recombinant IL-15 and IL-21,
(b) induced expression of endogenous IL-15 and IL-21,
(c) both (a) and (b), or
(d) recombinant IL-15 or IL-21, and induced expression of endogenous IL-21 or IL-15, respectively,
wherein following the modifying step the T cells are protected from apoptosis upon exposure to cancer cells.

45. A method of increasing the expansion and persistence of T cell therapy, comprising the step of modifying the T cells to express:
(a) recombinant IL-15 and IL-21,
(b) induced expression of endogenous IL-15 and IL-21,
(c) both (a) and (b), or
(d) recombinant IL-15 or IL-21, and induced expression of endogenous IL-21 or IL-15, respectively,
wherein following the modifying step the T cells have increased expansion and persistence compared to T cells without the modifying.


SEQUENCE LISTING (10 sequences)

SEQ ID NO: 1 - protein, 261 aa, Artificial Sequence
  Description of Artificial Sequence Synthetic polypeptide
MDWIWRILFLVGAATGAHSQVQLQQSGAELVRPGASVKLSCKASGYTFTDYEMHWVKQTPVHGLKWIGALDPKTGDTAYSQKFKGKATLTADKSSSTAYMELRSLTSEDSAVYYCTRFYSYTYWGQGTLVTVSAGGGGSGGGGSGGGGSDVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQNTHVPPTFGSGTKLEIK

SEQ ID NO: 2 - protein, 814 aa, Artificial Sequence
  Description of Artificial Sequence Synthetic polypeptide
MERIVICLMVIFLGTLVHKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDSRAEGRGSLLTCGDVEENPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSRAEGRGSLLTCGDVEENPGPMDWIWRILFLVGAATGAHSQVQLQQSGAELVRPGASVKLSCKASGYTFTDYEMHWVKQTPVHGLKWIGALDPKTGDTAYSQKFKGKATLTADKSSSTAYMELRSLTSEDSAVYYCTRFYSYTYWGQGTLVTVSAGGGGSGGGGSGGGGSDVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQNTHVPPTFGSGTKLEIKEPKSCDKTHTCPPCPDPKFWVLVVVGGVLACYSLLVTVAFIIKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 3 - DNA, 27 bp, Artificial Sequence
  Description of Artificial Sequence Synthetic oligonucleotide
atcctctagactgccatggaacggatc

SEQ ID NO: 4 - DNA, 24 bp, Artificial Sequence
  Description of Artificial Sequence Synthetic oligonucleotide
cgtccctcggctctagaatcttcg

SEQ ID NO: 5 - DNA, 20 bp, Artificial Sequence
  Description of Artificial Sequence Synthetic oligonucleotide
tagagccgagggacggggct

SEQ ID NO: 6 - DNA, 27 bp, Artificial Sequence
  Description of Artificial Sequence Synthetic oligonucleotide
atgatgacgcgttaatcatctgggggg

SEQ ID NO: 7 - DNA, 34 bp, Artificial Sequence
  Description of Artificial Sequence Synthetic oligonucleotide
atcctctagactgccatgagaatcagcaagcccc

SEQ ID NO: 8 - DNA, 27 bp, Artificial Sequence
  Description of Artificial Sequence Synthetic oligonucleotide
atgatgacgcgttaatcatctgggggg

SEQ ID NO: 9 - DNA, 27 bp, Artificial Sequence
  Description of Artificial Sequence Synthetic oligonucleotide
atcctctagactgccatggaacggatc

SEQ ID NO: 10 - DNA, 30 bp, Artificial Sequence
  Description of Artificial Sequence Synthetic oligonucleotide
cagtgcggccgctcaggccctgctggtgtt